SEQUENCE LISTING (22 sequences)

SEQ ID NO: 1 - DNA, 5425 bp, Rattus norvegicus
ggatcctcaggcagttcaatgtccaattttctgaggaacctccagactgatttccagaatggttttaccagtctgcaatcccaccaacaatggaggagtgttcctctttctccacatcctcgccagcatctgctgtcacctgagtttttgatcttagccattctcactggtgtgaggtgaaatctcagggttgttttgatttgcatttcccttatgactaaagatgttgaacatttctttaggtgtttctcagccattcggcattcctcagctgtgaattctttgtttagctctgaaccccattttttaatagggttatttgtttccctgcggtctaacttcttgagttctttgtatattttggatataaggcctctatctgttgtaggattggtaaagatcttttcccaatctgttggttgccgttttgtcctaaccacagtgtcctttgccttacagaagctttgcagttttatgagatcccatttatcgattcttgatcttggagcataagccattggtgttttgttcaggaaattttttccagtgcctatgtgttccagatgcttccctagtttttcttctattagtttgagtgtgtctggtttgatgtggaggtccatgatccacttggacttaagctttgtacagggtgataagcatggatcgatctgcattcttctacatgttgccctccagttgaaccagcaccatttgctgaaaatgctatcttttttccattggatggttttggctcctttgtcaaaaatcaagtgaccataggtgtgtgggttcatttctgggtcttcaattctattccattggtctatctgtctgtctctgtaccaataccatgcagttttatcaccattgctctgtaatactgcttgagttcagggatagtgattccccctgaagtccttttattgttgaggatagctttagctatcctgggttttttgttattccagatgaatttgcaaattgttctgtctaactctttgaagaattggattggtattttgatggggattgcattgaatctgtagattgcttttggtaaaatggccatttttactatattaatcctgccaatccatgagcatgggagatctttccatcttctgaggtcttcttcaatttctttcctcagtgtcttgaagttcttattgtacagatcttttacttgcttggttaaagtcacaccgaggtactttatattatttgggtctattatgaagggtgtcatttccctaatttctttctcggcttgtttctcttttgtatagaggaaggcaactgatttatttgagttaattttatacccagccactttgctgaagttgtttatcagctttagtagttctctggtggaacttttgggatcacttaaatatactatcatgtcatctgcaaatagtgatattttgacctcttcttttccgatctgtatccctttgatctccttttgttgtctgattgctctggctagaacttcaagaactatattgaaaaagtagggagagagtgggcagccttgtctagtccctgattttagtgggattgcttcaagtttctctccatttagtttaatgttagcaactggtttgctgtatatggcttttactatgtttagtatgggccttgaattcctattctttccaggacttttatcatgaaggggtgttaaaacacgataatttaatggagatgtttatgtttgggaggaccttgagagaaaggcactggtccaagtcattatagaatcatagtctaagccaaaaatactcatatatgttcagtttcactaccctccttctcctcgagacgtgtgacctaagtagctgtctgtatacggtaatatacttgccagttcctgagataagcaggcgcacagtgccccattccgaaataagtgtgcagccagtgttctagctttagtctccgatggaatcccctggttcaagtgatgatctcatgttgtcgcatcccatttccttccagggagctccctcaggagaacgactgctcacccgcgacctttccacaggattgaagctcagggaaggaaacaatatttgaagtttcagtccagtggtgcatatgaactttagaatcactccaccactatctacaacgacaaaagtcacatggctaaagaagagtgtcagaaaagagctccttaaaaaataaaattcaattcaattcaattcttgagcagtcctagtctcgtccatcagccttctctctggtccctctaattctctgactcactctctgaccagtgtagccagtaaaggggacattttctctaaataaatgttttcttccaacttgaaacctaatgtttacaagcctttatattacagtaccttggtttctccactctgatgaaatacaaacatcttacctatcattcagtcatgggttttctttctttgttctcagctctctgggtacctgtcatggactgccacatgatgattggctgagcagtggtgaagacactaacctcactggaatcataaagtagatttctaattaagaaaaaggagtgccacccattgttcaaaggaacactgtgttaatttagttagagtggagggtgcaaattctaagaggaaagctaatttataaacacggtgccagaacctgtttcccaaagttctgttgtgccgggctagaaccacacatttgaggttgtagtcaatggctgagaacaggagagatcaagcacccccagaaaggtcctctttttgaacaaagattatcagtgcatccagttaaataagaaagttgacagtggttgagagaagcaaccaggtgcttgccagccccacataactccaggctagcagcaaagatgaccttactctcccgatacaagctgggtcacttacagtgctatacttcgctattttcctctactcataccctttagaaaactgccaaattgatatgttttccatacctataatcagactaagaaaaagaggcagaaacaagttttaaaagctttcatggatggcactggagaagttaaatttttttgctgtataacctatgtttattttataccataaggacttcacaaactaacttaacgttattggaccagtacaaacctactttctttaatggcttctgagttacttgaagacaagtaacgtgtctactttattatggcgcctacattggaacccactgcacatcaccagagatttttctcagcaaatgtttgttgacttaattgaatgctgtcattgacagaaagtctggtttgcatttcctctcaactccttgaaaatagctacctttcctaattgaccctgttggcagatgtttaaactggggtgaacgctgtgctactgaggcctgatgctttgtggaaaatcaattagaatgagaccaaatgttccacctaattcaaatggttgtcaaaaaatagaatttgagtatctatatttcgtcccagctgacccctgagtgggctatgggtttgtggaagtagagatagaggagaagggaccattacatgtcctgctgcctgagttctgctttccttctccctttgaaggtgagctggggtctcagctgagctaagaatccagctatcaatagaaactatgaaacagttccagggacaaagataccaggtccccaacaactgcaactttctgggaaatgaggtggaaaatgctcagccaaggaaaaagagggccttaccctctctgggacaatgattgtgctgtgaactgcttcatcacggcatctggccccttgttaataatctaattaccctaggtctaagtagagttgttgacgtccaatgagcgctttctgcagacttagcactaggcaagtgtttggaaattacagcttcagcccctctcgccatctgcctacctacccctcctagagcccttaatgggccaaacggcaaagtccagggggcagagaggaggtgctttggactataaagctagtggagacccagtaactcccaaccctaagtgaccagctacaatcatagaccatcagcaagcaggtatgtactctcctgggtgagcccggttcccccagccaaaactctagggactttaggaaggatgtgggttcctctcttacatggaccttttcctagcctcaaccctgcctatcttccaggtcattgttccaacatggccctgtggatgcgcttcctgcccctgctggccctgctcgtcctctgggagcccaagcctgcccaggcttttgtcaaacagcacctttgtggtcctcacctggtggaggctctgtacctggtgtgtggggaacgtggtttcttctacacacccaagtcccgtcgtgaagtggaggacccgcaagtgccacaactggagctgggtggaggcccggaggccggggatcttcagaccttggcactggaggttgcccggcagaagcgtggcattgtggatcagtgctgcaccagcatctgctccctctaccaactggagaactactgcaactgagtccaccactccccgcccacccctctgcaatgaataaagcctttgaatgagcaccaaaatgagagagtttttatgaatacaaagggattgtgtgaacgggaatctttttctctgtcatttagtatcgtgctagcgtattactaagcagttgttaaaactgcatgattgtgtaaccatttaagaagctcatgataaaacacgacataattcaaagtatccagaatttggataataagagagtcaaagggtaaatgccactgaagttactagtccaaaagtgatttgcttctgtcttggctcacccccctggtactatttagctcacaattccaggttacagttcatccacagaaggggagtggaatagagacggataaataaatgtacccatacatcaacctatctagatagtgtcccactgagaatccccttcctagattatcctagactatcaagtagacagttaaaacatcacaatcactttgcacatcccacaccatcctgcaataattcattcactttctcatatacatcagagagatcactccttccacagataactacagtggtaaaggtcagtgtgagttcctatacagctgtggagtgatacagtctgtgcccttaacactttggttccagacagaggaaggaggaataaacacgagaacacaggtatgcggtttgtaaaccagtacacgaatgtttagatttttaaaaagatttttgtgggccaactaaggagtgagtgtctgttgccagaaaatgtctttccaaccttggtgtgaacactgacccatcaacactagaatacaaggttctgacttccaggctttctaagtcagaactcctgttgtcacagctgtagctatgaatctaacaaaagctcttcagtcttcgtatgaataggatcc

SEQ ID NO: 2 - DNA, 4044 bp, Homo sapiens
ctcgaggggcctagacattgccctccagagagagcacccaacaccctccaggcttgaccggccagggtgtccccttcctaccttggagagagcagccccagggcatcctgcagggggtgctgggacaccagctggccttcaaggtctctgcctccctccagccaccccactacacgctgctgggatcctggatctcagctccctggccgacaacactggcaaactcctactcatccacgaaggccctcctgggcatggtggtccttcccagcctggcagtctgttcctcacacaccttgttagtgcccagcccctgaggttgcagctgggggtgtctctgaagggctgtgagcccccaggaagccctggggaagtgcctgccttgcctccccccggccctgccagcgcctggctctgccctcctacctgggctccccccatccagcctccctccctacacactcctctcaaggaggcacccatgtcctctccagctgccgggcctcagagcactgtggcgtcctggggcagccaccgcatgtcctgctgtggcatggctcagggtggaaagggcggaagggaggggtcctgcagatagctggtgcccactaccaaacccgctcggggcaggagagccaaaggctgggtgtgtgcagagcggccccgagaggttccgaggctgaggccagggtgggacatagggatgcgaggggccggggcacaggatactccaacctgcctgcccccatggtctcatcctcctgcttctgggacctcctgatcctgcccctggtgctaagaggcaggtaaggggctgcaggcagcagggctcggagcccatgccccctcaccatgggtcaggctggacctccaggtgcctgttctggggagctgggagggccggaggggtgtaccccaggggctcagcccagatgacactatgggggtgatggtgtcatgggacctggccaggagaggggagatgggctcccagaagaggagtgggggctgagagggtgcctggggggccaggacggagctgggccagtgcacagcttcccacacctgcccacccccagagtcctgccgccacccccagatcacacggaagatgaggtccgagtggcctgctgaggacttgctgcttgtccccaggtccccaggtcatgccctccttctgccaccctggggagctgagggcctcagctggggctgctgtcctaaggcagggtgggaactaggcagccagcagggaggggacccctccctcactcccactctcccacccccaccaccttggcccatccatggcggcatcttgggccatccgggactggggacaggggtcctggggacaggggtccggggacagggtcctggggacaggggtgtggggacaggggtctggggacaggggtgtggggacaggggtgtggggacaggggtctggggacaggggtgtggggacaggggtccggggacaggggtgtggggacaggggtctggggacaggggtgtggggacaggggtgtggggacaggggtctggggacaggggtgtggggacaggggtcctggggacaggggtgtggggacaggggtgtggggacaggggtgtggggacaggggtgtggggacaggggtcctggggataggggtgtggggacaggggtgtggggacaggggtcccggggacaggggtgtggggacaggggtgtggggacaggggtcctggggacaggggtctgaggacaggggtgtgggcacaggggtcctggggacaggggtcctggggacaggggtcctggggacaggggtctggggacagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccgccctaatgggccaggcggcaggggttgacaggtaggggagatgggctctgagactataaagccagcgggggcccagcagccctcagccctccaggacaggctgcatcagaagaggccatcaagcaggtctgttccaagggcctttgcgtcaggtgggctcagggttccagggtggctggaccccaggccccagctctgcagcagggaggacgtggctgggctcgtgaagcatgtgggggtgagcccaggggccccaaggcagggcacctggccttcagcctgcctcagccctgcctgtctcccagatcactgtccttctgccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcagggtgagccaaccgcccattgctgcccctggccgcccccagccaccccctgctcctggcgctcccacccagcatgggcagaagggggcaggaggctgccacccagcagggggtcaggtgcacttttttaaaaagaagttctcttggtcacgtcctaaaagtgaccagctccctgtggcccagtcagaatctcagcctgaggacggtgttggcttcggcagccccgagatacatcagagggtgggcacgctcctccctccactcgcccctcaaacaaatgccccgcagcccatttctccaccctcatttgatgaccgcagattcaagtgttttgttaagtaaagtcctgggtgacctggggtcacagggtgccccacgctgcctgcctctgggcgaacaccccatcacgcccggaggagggcgtggctgcctgcctgagtgggccagacccctgtcgccagcctcacggcagctccatagtcaggagatggggaagatgctggggacaggccctggggagaagtactgggatcacctgttcaggctcccactgtgacgctgccccggggcgggggaaggaggtgggacatgtgggcgttggggcctgtaggtccacacccagtgtgggtgaccctccctctaacctgggtccagcccggctggagatgggtgggagtgcgacctagggctggcgggcaggcgggcactgtgtctccctgactgtgtcctcctgtgtccctctgcctcgccgctgttccggaacctgctctgcgcggcacgtcctggcagtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactagacgcagcctgcaggcagccccacacccgccgcctcctgcaccgagagagatggaataaagcccttgaaccagccctgctgtgccgtctgtgtgtcttgggggccctgggccaagccccacttcccggcactgttgtgagcccctcccagctctctccacgctctctgggtgcccacaggtgccaacgccggccaggcccagcatgcagtggctctccccaaagcggccatgcctgttggctgcctgctgcccccaccctgtggctcagggtccagtatgggagcttcgggggtctctgaggggccagggatggtggggccactgagaagtgacttcttgttcagtagctctggactcttggagtccccagagaccttgttcaggaaagggaatgagaacattccagcaattttccccccacctagccctcccaggttctatttttagagttatttctgatggagtccctgtggagggaggaggctgggctgagggagggggt

SEQ ID NO: 3 - DNA, 17 bp, Artificial Sequence
  Synthetic Primer
gctgagctaagaatcca

SEQ ID NO: 4 - DNA, 16 bp, Artificial Sequence
  Synthetic Primer
ctgagcattttccacc

SEQ ID NO: 5 - DNA, 19 bp, Artificial Sequence
  Synthetic Primer
gggagttactgggtctcca

SEQ ID NO: 6 - DNA, 27 bp, Artificial Sequence
  Synthetic Primer
gcagaattcctgcttgctgatggtcta

SEQ ID NO: 7 - DNA, 19 bp, Artificial Sequence
  Synthetic Primer
gttggaacaatgacctgga

SEQ ID NO: 8 - DNA, 19 bp, Artificial Sequence
  Synthetic Primer
ggcagaaggacagtgatct

SEQ ID NO: 9 - DNA, 21 bp, Artificial Sequence
  Synthetic Primer
aacatgaccaaatcatacagc

SEQ ID NO: 10 - DNA, 21 bp, Artificial Sequence
  Synthetic Primer
tgaaactgaaactgacgtgcc

SEQ ID NO: 11 - DNA, 21 bp, Artificial Sequence
  Synthetic Primer
atggcgcctcatcccttggat

SEQ ID NO: 12 - DNA, 21 bp, Artificial Sequence
  Synthetic Primer
acacaagaagtctgagaacac

SEQ ID NO: 13 - DNA, 22 bp, Artificial Sequence
  Synthetic Primer
agcatgcagcaggacggtctca

SEQ ID NO: 14 - DNA, 21 bp, Artificial Sequence
  Synthetic Primer
ttatggccagtgtaagtaata

SEQ ID NO: 15 - DNA, 23 bp, Artificial Sequence
  Synthetic Primer
atgtcgctgaccaacacaaagac

SEQ ID NO: 16 - DNA, 23 bp, Artificial Sequence
  Synthetic Primer
tcaccaagtccactgctgggcct

SEQ ID NO: 17 - DNA, 21 bp, Artificial Sequence
  Synthetic Primer
gccaccatgaatagtgaggag

SEQ ID NO: 18 - DNA, 24 bp, Artificial Sequence
  Synthetic Primer
tcagcctgcggtcctcaccggggt

SEQ ID NO: 19 - DNA, 18 bp, Artificial Sequence
  Synthetic Primer
ctgtgggatgttagctgt

SEQ ID NO: 20 - DNA, 20 bp, Artificial Sequence
  Synthetic Primer
tcaggacgagccgtgggcct

SEQ ID NO: 21 - DNA, 20 bp, Artificial Sequence
  Synthetic Primer
atggccgcggagctggcgat

SEQ ID NO: 22 - DNA, 21 bp, Artificial Sequence
  Synthetic Primer
ctacaggaagaagtcggccgt

(19) United States
(12) Patent Application Publication
(10) Pub. No.: US 2014/0294924 A1
(43) Pub. Date: Oct. 2, 2014

(54) Regeneration of Pancreatic Islets and Reversal of Diabetes by Islet Transcription Factor Genes Delivered In Vivo

(71) Applicants: Paul A. Grayburn, Plano, TX (US); Shuyuan Chen, Allen, TX (US); Jiahuan Ding, Murphy, TX (US)

(72) Inventors: Paul A. Grayburn, Plano, TX (US); Shuyuan Chen, Allen, TX (US); Jiahuan Ding, Murphy, TX (US)

(73) Assignee: Baylor Research Institute, Dallas, TX (US)

(21) Appl. No.: 14/191,402

(22) Filed: Feb. 26, 2014

Related U.S. Application Data
(63) Continuation of application No. 13/128,840, filed on Aug. 3, 2011, now abandoned.
(60) Provisional application No. 61/114,407, filed on Nov. 13, 2008.

(51) Int. Cl. A61K 9/127; A61K 31/713

(52) U.S. Cl. CPC A61K 9/1271; A61K 31/713
USPC 424/450; 514/44 R; 435/375

(57) ABSTRACT

The present invention includes compositions and methods for regenerating glucose-responsive cells by ultrasound-targeted microbubble destruction in the pancreas, wherein the composition comprises a pre-assembled liposome-nucleic acid complex in contact with within and about a microbubble, wherein the pre-assembled liposome-nucleic acid complex comprises a NeuroD gene under the control of the promoter, wherein disruption of the microbubble in the pancreas at a target site delivers the nucleic acid into pancreas cells at the location of the ultrasound disruption, wherein cells that incorporate the nucleic acid express insulin in response to high blood glucose levels.

### CROSS-REFERENCE TO RELATED APPLICATIONS

This application is a continuation of U.S. application Ser. No. 13/128,840, filed Aug. 3, 2011, which is a national phase filing under U.S. §371 of PCT International Application Serial No. PCT/US2009/064467, filed Nov. 13, 2009, which claims priority to U.S. Provisional Application Ser. No. 61/114,407, filed Nov. 13, 2008, the contents all of which is applications are incorporated by reference herein in its their entirety. This application is related to U.S. Provisional Application Ser. No. 60/846,465, filed Sep. 22, 2006, and U.S. application Ser. No. 11/859,709, filed Sep. 21, 2007.

### STATEMENT OF FEDERALLY FUNDED RESEARCH

This invention was made with U.S. Government support under Contract No. P01DK58398 awarded by the NIH. The government has certain rights in this invention.

### TECHNICAL FIELD OF THE INVENTION

The present invention relates to treatments for diabetes, and more particularly, to compositions and methods for the regeneration of cells glucose-responsive, insulin producing cells.

### INCORPORATION-BY-REFERENCE OF MATERIALS FILED ON COMPACT DISC

None.

### BACKGROUND OF THE INVENTION

Without limiting the scope of the invention, its background is described with respect to diabetes. Diabetes affects approximately 200 million people worldwide and is increasing in prevalence. It is estimated to be the fifth leading cause of death in the world and results in serious complications, including cardiovascular disease, chronic kidney disease, blindness, and neuropathy.

Despite a wide variety of pharmacological treatments for diabetes, including insulin therapy, adequate blood sugar control is often difficult, in part because these agents are not able to duplicate the glucose regulatory function of normal islets. Accordingly, new treatment strategies have focused on replenishing the deficiency of beta cell mass common to both major forms of diabetes by either islet transplantation or beta-cell regeneration.

One promising opportunity in the area of diabetes therapy is taught by U.S. Pat. No. 6,232,288, issued to Kojima for composition for improving pancreatic function. Kojima teaches the use of the betacellulin protein itself, or a fragment thereof, to promote the differentiation of undifferentiated pancreatic cells into insulin-producing beta cells or pancreatic polypeptide producing F cells. The BTC protein composition improved glucose tolerance in patients and inhibited the growth of undifferentiated pancreatic cells. Methods for treating mammals, including humans, were also provided, however, long-term treatment of the diabetic condition was not achieved by providing the patient with the betacellulin protein intravenously.

### SUMMARY OF THE INVENTION

In one embodiment the present invention includes compositions and methods for ultrasound-targeted microbubble destruction in the pancreas comprising a pre-assembled liposome-nucleic acid complex in contact with within and about a microbubble, wherein the pre-assembled liposome-nucleic acid complex comprises a NeuroD gene under the control of the promoter, wherein disruption of the microbubble in the pancreas at a target site delivers the nucleic acid into pancreas cells at the location of the ultrasound disruption, wherein cells that incorporate the nucleic acid express insulin in response to high blood glucose levels. In one aspect, the composition further comprises one or more insulin responsive regulatory genes operatively linked to a high expression, regulatable insulin promoter region comprising: 50 contiguous bases of SEQ ID NO.: 1 in the region upstream of the transcriptional start site of NeuroD. In another aspect, the composition further comprises one or more genes selected from one or more insulin responsive regulatory genes operatively linked to an insulin promoter region selected from ngn3, GLP1, PDX1, Mafa, betacellulin, Nkx2.2, Nkx6.1, PAX4, Isl1, Cyclin D2 (and other members of the cyclin family), CDK4 (and other members of the cyclin dependent kinase family), and siRNAs against cyclin dependent kinase inhibitors, such as p16 and other members of the INK4 family or p27 and other members of the CIP/KIP family). In another aspect, the composition further comprises an agent that is co-administered with the composition, wherein the agent is selected from an anti-apoptotic agent, an anti-inflammatory agent, a JNK inhibitor, a GLP-1, a tacrolimus, a sirolimus, an anakinra, a Dervin polyamide or combinations thereof.

In another embodiment, the present invention includes a composition and method for regenerating pancreatic beta cells using ultrasound-targeted microbubble destruction in the pancreas of microbubbles comprising NeuroD in the pancreas. In one aspect, the NeuroD is a recombinant Neuro D. In another aspect, the composition further comprises comprises a NeuroD gene under the control of a CUBI, RIP2.1, RIP3.1 or HIP3.1 promoter, and the NeuroD is expressed in cells that have been targeted for expression by the ultrasound-targeted microbubble destruction.

Yet another embodiment of the present invention is a method for regenerating insulin responsive cells in vivo and in situ in a diabetic patient comprising: delivering an effective amount of to the pancreas, wherein cells in the pancreas causes the cell to secreted insulin in response to high glucose levels in the blood. In one aspect, effective amount of NeuroD in the pancreatic cells comprises delivering an exogenous nucleic acid segment that expresses a NeuroD gene. In another aspect, the Neuro D is delivered to the pancreas by ultrasound-targeted microbubble destruction. In yet another aspect, the effective amount of NeuroD in the pancreatic cells comprises delivering an exogenous nucleic acid segment that expresses a NeuroD gene under the control of a CUBI, RIP2.1, RIP3.1 or HIP3.1 promoter.

Another embodiment of the present invention is a method of making a target cell insulin responsive comprising: making a nucleic acid segment comprising a NeuroD gene under the control of an insulin responsive promoter selected from CUBI, RIP2.1, RIP3.1 or HIP3.1 promoter; loading the nucleic acid segment into a microbubble; injecting a patient with the microbubble; delivering the nucleic acid segment into a pancreatic cell; and maintaining the target cell under conditions effective to express the insulin responsive regulatory gene; wherein expression of the NeuroD in the target cell causes the cell to respond to high blood glucose. In one aspect, the method further comprises delivering to the pancreas one or more genes selected from PDX1, Nkx2.2, Nkx6.1, PAX4, MafA, ngn3 and combinations thereof under the control of the promoter. In another aspect, the method further comprises delivering an agent that is co-administered with the composition, wherein the agent is selected from an anti-apoptotic agent, an anti-inflammatory agent, a JNK inhibitor, a GLP-1, a tacrolimus, a sirolimus, an anakinra, a Dervin polyamide or combinations thereof. In one aspect, the microbubble comprises a pre-assembled liposome-nucleic acid complex liposomes. In another aspect, the microbubble comprises a pre-assembled liposome-nucleic acid complex liposomes that comprises 1,2-dipalmitoyl-sn-glycero-3-phosphatidylcholine and 1,2-dipalmitoyl-sn-glycero-3-phosphatidylethanolamine glycerol mixed with a plasmid.

In yet another embodiment, the present invention includes a method of restoring insulin responsiveness comprising the steps of: obtaining an isolated nucleic acid segment comprising one or more insulin responsive regulatory genes operatively linked to a high expression insulin promoter region a genomic fragment of the insulin promoter comprising a 5′ untranslated region, exon1, intron1 and exon2 of the insulin gene; transferring the nucleic acid segment into a target cell; and maintaining the target cell under conditions effective to express the insulin responsive regulatory gene; wherein expression of the insulin responsive regulatory gene in the target cell causes the cell to respond to high blood glucose. In one aspect, the insulin responsive cell is in an animal. In another aspect, the one or more insulin responsive regulatory genes are operatively linked to an insulin promoter region is in a viral or plasmid vector. In another aspect, one or more insulin responsive regulatory genes operatively linked to an insulin promoter region are selected from NeuroD, ngn3, GLP1, PDX1, Mafa, betacellulin, Nkx2.2, Nkx6.1, PAX4, Isl1, Cyclin D2 (and other members of the cyclin family), CDK4 (and other members of the cyclin dependent kinase family), and siRNAs against cyclin dependent kinase inhibitors, such as p16 and other members of the INK4 family or p27 and other members of the CIP/KIP family).

Another embodiment of the present invention is a method of restoring insulin responsiveness comprising the steps of: obtaining an isolated nucleic acid segment comprising one or more insulin responsive regulatory genes operatively linked to an insulin promoter region comprising a genomic fragment of the insulin promoter comprising a 5′ untranslated region, exon1, intron1 and exon2 of the insulin gene; transferring the nucleic acid segment into a pancreatic cell; and maintaining the target cell under conditions effective to express the insulin responsive regulatory gene; wherein expression of the insulin responsive regulatory gene in the target cell causes the cell to respond to high blood glucose. In one aspect, the insulin promoter region comprises 100 to 500 contiguous bases of SEQ ID NO.: 1 in the region upstream of the transcriptional start site. In one embodiment, the insulin promoter region comprises the entire region upstream of the transcriptional start site in SEQ ID NO.: 1, or even 100 to 500 contiguous bases of SEQ ID NO.: 1 in the region upstream of the transcriptional start site. Another aspect is an isolated nucleic acid comprising a high expression insulin promoter region comprising: 50 contiguous bases of SEQ ID NO.: 1 in the region upstream of the transcriptional start site for one or more insulin responsive genes.

In another embodiment, the present invention is a composition for ultrasound-targeted microbubble destruction in the pancreas comprising: a pre-assembled liposome-nucleic acid complex in contact with a microbubble, wherein the pre-assembled liposome-nucleic acid complex comprises comprising one or more insulin responsive regulatory genes operatively linked to a high expression, regulatable insulin promoter region comprising: a genomic fragment of the insulin promoter comprising a 5′ untranslated region, exon1, intron1 and exon2 of the insulin gene, wherein ultrasound disruption of the microbubble in the pancreas at a target site delivers the nucleic acid into pancreas cells at the location of the ultrasound disruption. In one aspect, the pre-assembled liposome-nucleic acid complex comprises cationic lipids, anionic lipids or mixtures and combinations thereof. In another aspect, the microbubbles are disposed in a pharmaceutically acceptable vehicle. In another aspect, active agent nucleic acid comprises an insulin gene. In one aspect, the active agent nucleic acid comprises a nucleic acid vector that comprises a hexokinase gene under the control of the promoter. In another aspect, the active agent nucleic acid comprises a nucleic acid vector that comprises a NeuroD gene under the control of the promoter. The pre-assembled liposome-nucleic acid complex liposomes can be, e.g., 1,2-dipalmitoyl-sn-glycero-3-phosphatidylcholine and 1,2-dipalmitoyl-sn-glycero-3-phosphatidylethanolamine glycerol mixed with a plasmid. In another aspect, the composition may further comprise a coating. In another aspect, the composition may further comprise one or more insulin responsive regulatory genes operatively linked to an insulin promoter region are selected from NeuroD, ngn3, GLP1, PDX1, Mafa, betacellulin, Nkx2.2, Nkx6.1, PAX4, Isl1, Cyclin D2 (and other members of the cyclin family), CDK4 (and other members of the cyclin dependent kinase family), and siRNAs against cyclin dependent kinase inhibitors, such as p16 and other members of the INK4 family or p27 and other members of the CIP/KIP family).

A cell made insulin responsive by a method comprising injecting into a cell a pre-assembled liposome-nucleic acid microbubble complex, wherein the pre-assembled liposome-nucleic acid complex comprises a NeuroD gene under the control of an insulin promoter comprising one or more insulin responsive regulatory genes operatively linked to an insulin promoter region comprising: a genomic fragment of the insulin promoter comprising a 5′ untranslated region, exon1, intron1 and exon2 of the insulin gene, wherein disruption of the microbubble in the pancreas at a target site delivers the nucleic acid into pancreas cells at the location of the ultrasound disruption, wherein cells that incorporate the nucleic acid express insulin in response to high blood glucose levels. In one aspect, the cell further comprises one or more insulin responsive regulatory genes operatively linked to a regulatable insulin promoter region comprising 50 contiguous bases of region upstream of the insulin start site upstream from a NeuroD gene. In another aspect, the cell further comprises one or more genes selected from one or more insulin responsive regulatory genes operatively linked to an insulin promoter region selected from ngn3, GLP1, PDX1, Mafa, betacellulin, Nkx2.2, Nkx6.1, PAX4, Isl1, Cyclin D2 (and other members of the cyclin family), CDK4 (and other members of the cyclin dependent kinase family), and siRNAs against cyclin dependent kinase inhibitors, such as p16 and other members of the INK4 family or p27 and other members of the CIP/KIP family).

### BRIEF DESCRIPTION OF THE DRAWINGS

The accompanying figures, in which like reference numerals refer to identical or functionally-similar elements throughout the separate views and which are incorporated in and form part of the specification, further illustrate the present invention and, together with the detailed description of the invention, serve to explain the principles of the present invention.

FIG. 1 is a schematic representation of the rat insulin promoter area and exon1, intron1 and exon2. The rat insulin promoter is shown with known sequence elements and fusion exon1 and exon2;

FIG. 2A-2B: Top panel are luciferase activity of INS-1 cells lyses 48 hours after transfected with rips-luc under three different glucose concentration. Bottom panel are luciferase activity of culture media solution in different culture time after transfection with rips-luc under high glucose concentration, no luciferase activity in no glucose and in normal glucose concentration (data not shown);

FIG. 3A-3F: RIP3.1-DsRed slides, top left:green as anti-insulin; top middle red as anti-dsred; and top right as their confocal image. A bottom as sequential section and similar islets structure, bottom left: green as anti-glucagon; bottom middle:red as anti-dsred.bottom right as their confocal image. (3B): RIP-4.1-dsred; (3C): RIP-1.1-dsred; (3D): RIP-1.1-dsred slides, (3E): pCMV-dsred; F: normal control;

FIG. 4A-4B: images as pRIP3.1-DsRed rats with 10% glucose feeding, A top right Green as anti-insulin; A top middle red as anti-dsred, A top left as their confocal image; A bottom right green as anti-glucagon; A bottom middle red as anti-dsred; A bottom left:their confocal image; (4B) images as pRIP3.1-DsRed rats fasting overnight;

FIG. 5: Top panels. Microscopic sections (400×) from a control rat (left) and a UTMD-treated rat (middle as feeding rat and right as fasting rat). In-situ PCR was used to stain for the DsRed plasmid DNA, which is seen throughout the treated pancreas. An islet is clearly seen (arrows). Bottom panels. Sections (400×) from a control rat (left) and a rat treated with UTMD using RIP6.1-DsRed (middle as feeding rat and right as fasting rat). In-situ RT-PCR was used to stain for DsRed mRNA, which is localized to the islet center (middle/feeding). Stained at islets border (right/fasting rat);

FIG. 6 shows the results of immunofluorescent microscopy. The top panels show representative examples of islets from the 30-day experiments. FITC-labeled anti-insulin (green) demonstrates beta-cells, whereas DsRed-labeled anti-glucagon demonstrates alpha cells. Ultrasound targeted gene therapy with Nkx2.2, Nkx6.1, Pax4, Ngn3, and MafA resulted in formation of alpha-cell dominant islets. In contrast, NeuroD1-treated rats had nearly normal islet architecture with central beta-cells surrounded by peripheral alpha cells. The bottom left panel shows the islet count per slide for the different groups. Both normal controls and NeuroD1-treated rats had significantly more islets per slide than all other groups (*p<0.0001 by ANOVA). The bottom right panel shows the number of beta-cells per islet. Both normal controls and NeuroD1-treated rats had significantly higher percentage of beta-cells per islet than all other groups (*p<0.0001 by ANOVA);

FIG. 7A-7B shows the results of blood glucose (right upper panel), blood insulin (right lower panel), and C-peptide (right upper panel) in the 30-day experiment. At day 3, all STZ-treated rats had markedly elevated blood glucose and decreased insulin and C-peptide relative to normal controls (p<0.0001). However, by day 30, only the NeuroD1-treated rats had restoration of blood glucose, insulin, and C-peptide to normal or nearly normal levels. The bottom right panel shows the results of glucose tolerance tests in a separate group of 6 NeuroD1-treated rats (n=6) compared to normal controls (n=3) and DsRed-treated rats (n=3). Gene therapy with NeuroD1 after STZ-induced diabetes resulted in restoration of glucose tolerance to normal;

FIG. 8 shows markers of cell proliferation. The top left and middle panels show islets co-stained with FICT-labeled anti-insulin (green), and DsRed-labeled anti-BrdU (left) and anti-Ki67 (middle). The top right panel shows the number of Ki67 positive, insulin positive cells, which is statistically significantly higher in NeuroD1-treated STZ rats than in normal controls, STZ-treated control rats, or STZ-treated rats treated with DsRed by UTMD (p<0.0001). The bottom panels show an islet from a NeuroD1-treated STZ-rat stained with anti-Ck19 (left, green), anti-insulin (left center, red), anti-ngn3 (blue, right center), and their confocal image (right). Insulin positive beta-cells co-stain with ngn3, but not with Ck19, indicating that the regenerated islets are not of ductal cell origin;

FIG. 9A-9D show images of representative islets from an experiment in which rats were treated with various genes and combinations of genes by UTMD. Triple staining with DAPI (blue stain for nuclei), anti-insulin (green), and anti-glucagon (red). CyclinD2, CDK4, and GLP 1 (islets were stable up to 180 days) when treated with the combination. The upper left panel shows a representative islet from a normal control rat not treated by UTMD. A large dense islet core of beta cells expressing insulin is present (green) surrounded by a small capsule of peripheral alpha cells expressing glucagon (red). The upper right panel shows a representative islet remnant after STZ-induced diabetes. Only a few beat cells are present. The bottom left panel shows an example of islet regeneration after UTMD with the GLP1 gene. A smaller than normal islet is present with some beta cells (green) and alpha cells (red), but the architecture is not normal. Similar findings were present (not shown) for rats treated with UTMD using the single genes CyclinD2, CDK4, and CDK6. The bottom right panel shows a nearly normal islet after UTMD with the combination of CyclinD2, CDK4, and GLP1 (these islets were stable up to 180 days and were accompanied by reversal of diabetes with normal blood glucose, insulin and C-peptide levels);

FIG. 10 is a plot showing blood glucose levels over time of islets in various groups of rats treated with UTMD gene therapy, as well as normal controls, and STZ diabetic rats without UTMD treatment. As can be seen, single gene therapy with CyclinD2, CDK4, CDK6, or GLP1 did not result in normalization of blood glucose. However, the composition comprising a combination CyclinD2, CDK4, and GLP1, or CyclinD2, CDK4, CDK6, and GLP1 restored blood glucose to normal levels for 4 weeks in this particular experiment. Longer term studies in another group of animals confirmed a duration of effect of up to 180 days;

FIG. 11 is a map of the HIP-hNeuroD1 plasmid;

FIG. 12 is a map of the RIP3.1-DsRed plasmid;

FIG. 13 is a map of the RIP-DsRed 4.1 plasmid;

FIG. 14 is a map of the RIP-DsRed 5.1 plasmid; and

FIG. 15 is a map of the RIP-DsRed 2.1 plasmid.

### DETAILED DESCRIPTION OF THE INVENTION

The novel features of the present invention will become apparent to those of skill in the art upon examination of the following detailed description of the invention. It should be understood, however, that the detailed description of the invention and the specific examples presented, while indicating certain embodiments of the present invention, are provided for illustration purposes only because various changes and modifications within the spirit and scope of the invention will become apparent to those of skill in the art from the detailed description of the invention and claims that follow.

The present invention may include modifications and variations of each are possible in light of the teachings described herein without departing from the spirit and scope of the following claims. It is contemplated that the use of the present invention can involve components having different characteristics. It is intended that the scope of the present invention be defined by the claims appended hereto, giving full cognizance to equivalents in all respects.

As used herein, the terms “a sequence essentially as set forth in SEQ ID NO. (#)”, “a sequence similar to”, “nucleotide sequence” and similar terms, with respect to nucleotides, refer to sequences that substantially correspond to any portion of the sequence identified herein as SEQ ID NO.: 1. These terms refer to synthetic as well as naturally-derived molecules and includes sequences that possess biologically, immunologically, experimentally, or otherwise functionally equivalent activity, for instance with respect to hybridization by nucleic acid segments, or the ability to encode all or portions of NeuroD activity. Naturally, these terms are meant to include information in such a sequence as specified by its linear order.

As used herein, the term “gene” refers to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

As used herein, the term “vector” refers to nucleic acid molecules that transfer DNA segment(s) from one cell to another. The vector may be further defined as one designed to propagate specific sequences, or as an expression vector that includes a promoter operatively linked to the specific sequence, or one designed to cause such a promoter to be introduced. The vector may exist in a state independent of the host cell chromosome, or may be integrated into the host cell chromosome

As used herein, the term “host cell” refers to cells that have been engineered to contain nucleic acid segments or altered segments, whether archeal, prokaryotic, or eukaryotic. Thus, engineered, or recombinant cells, are distinguishable from naturally occurring cells that do not contain recombinantly introduced genes through the hand of man.

As used herein, the term “control sequences” refers to DNA sequences necessary for the expression of an operably linked coding sequence in a particular host organism. The control sequences that are suitable for prokaryotes, for example, include a promoter, optionally an operator sequence, a ribosome binding site, and transcriptional terminators. Highly regulated inducible promoters that suppress Fab′ polypeptide synthesis at levels below growth-inhibitory amounts while the cell culture is growing and maturing, for example, during the log phase may be used.

As used herein, the term “operably linked” refers to a functional relationship between a first and a second nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it effects the transcription of the sequence; or a ribosome binding site is operably linked to e coding sequence if it is positioned so as to facilitate translation. Generally, “operably linked” means that the DNA sequences being linked are contiguous and, in the case of a secretory leader, contiguous and in same reading frame. Enhancers do not have to be contiguous. Linking is accomplished by ligation at convenient restriction sites. If such sites do not exist, then synthetic oligonucleotide adaptors or linkers are used in accord with conventional practice.

As used herein, the term “cell” and “cell culture” are used interchangeably end all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Different designations are will be clear from the contextually clear.

As used herein, “Plasmids” are designated by a lower case p preceded and/or followed by capital letters and/or numbers. Starting plasmids may be commercially available, are publicly available on an unrestricted basis, or can be constructed from such available plasmids in accord with published procedures. In addition, other equivalent plasmids are known in the art and will be apparent to the ordinary artisan.

As used herein, the terms “protein”, “polypeptide” or “peptide” refer to compounds comprising amino acids joined via peptide bonds and are used interchangeably.

As used herein, the term “endogenous” refers to a substance the source of which is from within a cell. Endogenous substances are produced by the metabolic activity of a cell. Endogenous substances, however, may nevertheless be produced as a result of manipulation of cellular metabolism to, for example, make the cell express the gene encoding the substance.

As used herein, the term “exogenous” refers to a substance the source of which is external to a cell. When referring to nucleic acids, “exogenous” refers to a nucleic acid sequence that is foreign to the cell, or homologous to the cell but in a position within the host cell nucleic acid in which the element is ordinarily not found. An exogenous substance may nevertheless be internalized by a cell by any one of a variety of metabolic or induced means known to those skilled in the art.

A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed, excised or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotides referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand.

As used herein, the term “transformation,” refers to a process by which exogenous DNA enters and changes a recipient cell, e.g., one or more plasmids that include promoters and coding sequences to express NeuroD, ngn3, GLP1, PDX1, Mafa, betacellulin, Nkx2.2, Nkx6.1, PAX4, Isl1, Cyclin D2 (and other members of the cyclin family). CDK4 (and other members of the cyclin dependent kinase family), and siRNAs against cyclin dependent kinase inhibitors, such as p16 and other members of the INK4 family or p27 and other members of the CIP/KIP family). It may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome.

As used herein, the term “transfection” refers to the introduction of foreign DNA into eukaryotic cells. Transfection may be accomplished by a variety of means known to the art including, e.g., calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Thus, the term “stable transfection” or “stably transfected” refers to the introduction and integration of foreign DNA into the genome of the transfected cell. The term “stable transfectant” refers to a cell which has stably integrated foreign DNA into the genomic DNA. The term also encompasses cells which transiently express the inserted DNA or RNA for limited periods of time. Thus, the term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells which have taken up foreign DNA but have failed to integrate this DNA.

As used herein, the term “vector” is used in reference to nucleic acid molecules that transfer DNA segment(s) from one cell to another. The term “vehicle” is sometimes used interchangeably with “vector.” The term “vector” as used herein also includes expression vectors in reference to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “amplify” when used in reference to nucleic acids, refers to the production of a large number of copies of a nucleic acid sequence by any method known in the art. Amplification is a special case of nucleic acid replication involving template specificity. Template specificity is frequently described in terms of “target” specificity. Target sequences are “targets” in the sense that they are sought to be sorted out from other nucleic acid. Amplification techniques have been designed primarily for this sorting out.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer may be single stranded for maximum efficiency in amplification but may alternatively be double stranded. If double stranded, the primer is first treated to separate its strands before being used to prepare extension products. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g. ELISA, as well as enzyme-based histochemical assays), fluorescent, radioactive, and luminescent systems. It is not intended that the present invention be limited to any particular detection system or label.

As used herein, the term “target” when used in reference to the polymerase chain reaction, refers to the region of nucleic acid bounded by the primers used for polymerase chain reaction. Thus, the “target” is sought to be sorted out from other nucleic acid sequences. A “segment” is defined as a region of nucleic acid within the target sequence. A target when used in reference to a cell or tissue, refers to the targeting using a vector (e.g., a virus, a liposome or even naked nucleic acids) that are exogenous to a cell to deliver the nucleic acid into the cell such that it changes the function of the cell, e.g., expresses one or more BTC or PDX1 genes.

As used herein, the term “polymerase chain reaction” (“PCR”) refers to the method of K. B. Mullis U.S. Pat. Nos. 4,683,195, 4,683,202, and 4,965,188, hereby incorporated by reference, which describe a method for increasing the concentration of a segment of a target sequence in a mixture of genomic DNA without cloning or purification. This process for amplifying the target sequence includes a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as DCTP or DATP, into the amplified segment). In addition to genomic DNA, any oligonucleotide sequence can be amplified with the appropriate set of primer molecules. In particular the amplified segments created by the PCR process itself are, themselves, efficient templates for subsequent PCR amplifications.

As used herein, the term “staining reagent” refers to the overall hybridization pattern of the nucleic acid sequences that comprise the reagent. A staining reagent that is specific for a portion of a genome provides a contrast between the target and non-target chromosomal material. A number of different aberrations may be detected with any desired staining pattern on the portions of the genome detected with one or more colors (a multi-color staining pattern) and/or other indicator methods.

As used herein, the term “transgene” refers to genetic material that may be artificially inserted into a mammalian genome, e.g., a mammalian cell of a living animal. The term “transgenic animal is used herein to describe a non-human animal, usually a mammal, having a non-endogenous (i.e., heterologous) nucleic acid sequence present as an extrachromosomal element in a portion of its cells or stably integrated into its germ line DNA (i.e., in the genomic sequence of most or all of its cells). Heterologous nucleic acid is introduced into the germ line of such transgenic animals by genetic manipulation of, for example, embryos or embryonic stem cells of the host animal according to methods well known in the art.

As used herein, the term “transgene” refers to such heterologous nucleic acid, e.g., heterologous nucleic acid in the form of, e.g., an expression construct (e.g., for the production of a “knock-in” transgenic animal) or a heterologous nucleic acid that upon insertion within or adjacent a target gene results in a decrease in target gene expression (e.g., for production of a “knock-out” transgenic animal). A “knock-out” of a gene means an alteration in the sequence of the gene that results in a decrease of function of the target gene, preferably such that target gene expression is undetectable or insignificant. Transgenic knock-out animals include a heterozygous knock-out of a target gene, or a homozygous knock-out of a target gene.

As used herein, the term “stem cell” refers to totipotent or pluripotent stem cells, e.g., embryonic stem cells, and to such pluripotent cells in the very early stages of embryonic development, including but not limited to cells in the blastocyst stage of development. In one specific example for use with the present invention, the stem cell may be a pancreatic cell precursor that has not differentiated into an acinar or beta cell and is used as a target to express NeuroD, ngn3, GLP1, PDX1, Mafa, betacellulin, Nkx2.2, Nkx6.1, PAX4, Isl1, Cyclin D2 (and other members of the cyclin family). CDK4 (and other members of the cyclin dependent kinase family), and siRNAs against cyclin dependent kinase inhibitors, such as p16 and other members of the INK4 family or p27 and other members of the CIP/KIP family).

In a prior patent application, the present inventors demonstrated that gene therapy could be targeted to pancreatic islets in normal rats, using ultrasound targeted microbubble destruction (UTMD). Intravenous microbubbles carrying plasmid DNA are selectively destroyed within the pancreatic microcirculation by ultrasound, thereby locally delivering the plasmids. Islet specificity was achieved by incorporating the rat insulin-I promoter within the plasmid DNA. It has now been found that using UTMD can be used to deliver betacellulin (BTC), alone and in combination with PDX1 in streptozotocin (STZ)-induced diabetes in rats. Transformation of the target cells led to primitive islet-like clusters of glucagons-staining cells were seen in the rats treated with BTC and PDX1. In this study the clusters disappeared by 30 days after treatment. Although regeneration of normal islets was not seen, diabetes was reversed for up to 15 days after UTMD by transformation of pancreatic acinar cells into insulin-producing cells with beta-cell markers.

Diabetes mellitus is increasing in prevalence, affecting more than 5% of the population throughout the world. Novel therapeutic strategies, including new medications, islet transplantation, and gene therapy, are vigorously being sought to treat diabetes. Direct in vivo pancreatic gene delivery targeting the islets is a key approach for diabetic gene therapy. So far studies showed that the adenovirus, adeno-associated virus, lentivirus, and herpes simplex virus-1 vectors have rendered efficient gene transfer to the islets in vivo, but suffered from host immune responses and vector cytotoxicity. Non-viral gene delivery systems, including naked DNA and DNA complexes, have also demonstrated islet cell transfection at much lower levels as has transient transgene expression. However, it seems likely that non-viral vector systems will more easily satisfy biosafety concerns in clinical trials.

Ultrasound-targeted microbubble destruction (UTMD) has been used to deliver genes or drugs to pancreatic islets in vivo. Briefly, genes are incorporated into cationic liposomes and then attached to the phospholipid shell of gas-filled microbubbles, which are then injected intravenously and destroyed within the microcirculation of intact islets by ultrasound. The UTMD approach enables transfection of the entire islet core, where most beta cells reside. UTMD has been combined with a rat insulin promoter (RIP) to enhance beta-cell specificity. The present invention improves greatly the differential efficiency of gene expression by varying the length of the RIP segments. Non-coding regions in exon1 and exon2 of insulin gene were fused to provide enhanced downstream gene function in plasmids. At the transcriptional level, fusion of insulin mRNA upregulated downstream gene expression to such a high level that they mimicked normal insulin mRNA level in normal islet beta cells. UTMD was then used to deliver the insulin gene fusion plasmid under control of the rat insulin promoter driving to intact islets of adult, living animals, thereby providing a safe, tissue-specific, highly efficient and regulated gene expression for diabetic gene therapy.

### New Promoters.

Materials and Methods. Rat insulin promoters and plasmids constructs. Rat insulin gene 1 promoter fragments (RIP2.1 (−412 to −303); RIP1.1 (−412 to −1); RIP4.1 (−412 to +43); RIP3.1 (−412 to +165), GenBank Accession No. J00747, which is defined herein as SEQ ID NO.: 1, and GenBank Accession No. NC_{—}000011 for the human insulin promoter—defined as SEQ ID NO.: 2) both incorporated herein by reference), were amplified from Sprague-Dawley (SD) rat DNA using PCR. RIP forward primers (5′-G CTG AGC TAA GAA TCC A-3′) (SEQ ID NO.: 3); RIP2.1 reverse primer (5′-CTGAGC ATTTTCCACC-3′) (SEQ ID NO.: 4); RIP1.1 reverse primer (5′-GGGAGTTACTGGGTCTCCA-3′) (SEQ ID NO.: 5); RIP4.1 reverse primer (5′-GCAGAATTCCTGCTTGCTGATGGTCTA-3′) (SEQ ID NO.: 6) RIP3.1 reverse primer (5′-GTTGGAACAATGACCTGGA-3′) (SEQ ID NO.: 7); and reverse primer (5-GGCAGAAGGACAGTGATCT-3) (SEQ ID NO.: 8) containing a KpnI and xho1 restriction site, respectively. The sequences of these primers are listed in Table 1. The resulting DNA fragment was subcloned into the pGL2-basic firefly reporter plasmid and pDsRed1-1 reporter vector. SV40-promoter firefly luciferase reporter plasmid, pGL2-Control from Promega, pDsRed1-1 and pCMV-DsRed-express-1 from Clonetech. Rat genomic DNA was extracted from rat peripheral blood with a QIAamp Blood kit (Qiagen Inc, Valencia, Calif.) according to the manufacturer's instructions. The corresponding PCR products were verified by agarose gel electrophoresis and purified by QIAquick Gel Extraction kit (QIAGEN). To confirm the sequences, direct sequencing of PCR products was performed with dRhodamine Terminator Cycle Sequencing Kit (PE Applied Biosystems, Foster City, Calif.) on an ABI 3100 Genomic Analyzer. The plasmids digestion, ligation, subcloning, isolation and purification were performed by standard procedures, and once again sequenced to confirm that no artifactual mutations were present.

TABLE 1
Primer Sequences (rat insulin gene promoters
using the same forward primer).

| | | GenBank |
|---|---|---|
| | | Acces- |
| | | sion |
| Primer | Sequence | No. |
| RIP forward | 5′-g CTg AgC TAA gAA | J00747 |
| primers | TCC A | |
| | (SEQ ID NO.: 3) | |
| RIP2.1 reverse | 5′-CTg AgC ATT TTC | |
| primer | CAC C | |
| | (SEQ ID NO.: 4) | |
| RIP1.1 reverse | 5′-ggg AgT TAC Tgg gTC | |
| primer | TCC A | |
| | (SEQ ID NO.: 5) | |
| RIP4.1 reverse | 5′-CTg CTT gCT gAT | |
| primer | ggT CTA | |
| | (SEQ ID NO.: 6) | |
| RIP3.1 reverse | 5′-gAC CTg gAA gAT Agg | |
| primer | CAg ggT | |
| | (SEQ ID NO.: 7) | |
| Rat Neurod1 cDNA | 5′-AAC Atg ACC AAA TCA | NM_019218 |
| forward | TAC AgC | |
| | (SEQ ID NO.: 9) | |
| Rat Neurod1 cDNA | 5′-TgA AAC TgA AAC TgA | |
| reverse | CgT gCC | |
| | (SEQ ID NO.: 10) | |
| Rat Neurogenin3 | 5′-ATg gCg CCT CAT CCC | NM_021700 |
| cDNA forward | TTg gAT | |
| | (SEQ ID NO.: 11) | |
| Rat Neurogenin3 | 5′-ACA CAA gAA gTC TgA | |
| cDNA reverse | gAA CAC | |
| | (SEQ ID NO.: 12) | |
| Rat PAX4 cDNA | 5′-AgC ATg CAg CAg gAC | NM_031799 |
| forward | ggT CTCA | |
| | (SEQ ID NO.: 13) | |
| Rat PAX4 cDNA | 5′-TTA Tgg CCA gTg TAA | |
| reverse | gTA ATA | |
| | (SEQ ID NO.: 14) | |
| Rat NKX2.2 cDNA | 5′-ATg TCg CTg ACC AAC | XM_345446 |
| forward | ACA AAg AC | |
| | (SEQ ID NO.: 15) | |
| Rat NKX2.2 cDNA | 5′-TCA CCA AgT CCA CTg | |
| reverse | CTg ggC CT | |
| | (SEQ ID NO.: 16) | |
| Rat PDX1 cDNA | 5′-gCC ACC Atg AAT AgT | NM_022852 |
| forward | gAg gAg | |
| | (SEQ ID NO.: 17) | |
| Rat PDX1 cDNA | 5′-TCA gCC TgC ggT CCT | |
| reverse | CAC Cgg ggT | |
| | (SEQ ID NO.: 18) | |
| Golden hamster | 5′-CTg Tgg gAT gTT AgC | X81409 |
| NKX6.1 cDNA | TgT | |
| forward | (SEQ ID NO.: 19) | |
| Golden hamster | 5′-TCA ggA CgA gCC gTg | |
| NKX6.1 cDNA | ggC CT | |
| reverse | (SEQ ID NO.: 20) | |
| Human MafA cDNA | 5′-ATg gCC gCg gAg CTg | NM_201589 |
| forward | gCg AT | |
| | (SEQ ID NO.: 21) | |
| Human MafA cDNA | 5′-CTA CAg gAA gAA gTC | |
| reverse | ggC CgT | |
| | (SEQ ID NO.: 22) | |

Rat UTMD Protocol. Sprague-Dawley rats (250-350 g) were anesthetized with intraperitoneal ketamine (100 mg/kg) and xylazine (5 mg/kg). A polyethylene tube (PE 50, Becton Dickinson, MD) was inserted into the right internal jugular vein by cutdown. The anterior abdomen was shaved and an S3 probe (Sonos 5500, Philips Ultrasound, Andover, Mass.) placed to image the left kidney and spleen, which are easily identified. The pancreas lies between them, so the probe was adjusted to target the pancreas and clamped in place. One ml of microbubble solution was infused at a constant rate of 3 ml/h for 20 minutes using an infusion pump. Throughout the duration of the infusion, microbubble destruction was achieved using ultraharmonic mode (transmit 1.3 MHz/receive 3.6 MHz) with a mechanical index of 1.2-1.4 and a depth of 4 cm. The ultrasound pulses were ECG-triggered (at 80 ms after the peak of the R wave) to deliver a burst of 4 frames of ultrasound every 4 cardiac cycles. These settings have previously been shown to be the optimal ultrasound parameters for gene delivery using UTMD. At the end of each delivery the jugular vein was tied off and the skin closed. All rats were monitored after delivery for normal behavior. Rats were sacrificed 4 days later and the pancreas was harvested.

Manufacture of Plasmid-Containing Lipid-Stabilized Microbubbles. Lipid-stabilized microbubbles were prepared.^{5,6} Briefly, a solution of DPPC (1,2-dipalmitoyl-sn-glycero-3-phosphatidylcholine, Sigma, St. Louis, Mo.) 2.5 mg/ml; DPPE (1,2-dipalmitoyl-sn-glycero-3-phosphatidylethanolamine, Sigma, St. Louis, Mo.) 0.5 mg/ml; and 10% glycerol was mixed with 2 mg of plasmid solution in a 2:1 ratio. Aliquots of 0.5 ml of this phospholipid-plasmid solution were placed in 1.5 ml clear vials; the remaining headspace was filled with perfluoropropane gas (Air Products, Inc, Allentown, Pa.). Each vial was incubated at 40° C. for 30 min and then mechanically shaken for 20 seconds by a dental amalgamator (Vialmix™, Bristol-Myers Squibb Medical Imaging, N. Billerica, Mass.). The lipid-stabilized microbubbles appear as a milky white suspension floating on the top of a layer of liquid containing unattached plasmid DNA. The subnatant was discarded and the microbubbles washed three times with PBS to removed unattached plasmid DNA. The mean diameter and concentration of the microbubbles in the upper layer were measured by a particle counter (Beckman Coulter Multisizer III).

In Situ-PCR for Detection of DsRed DNA. DsRed Primers. A single pair of DsRed primers were used directed against the DsRed DNA; they are DsRed 125⁺ (5′-GAGTTCATGCGCTTCAAGGTG-3′) and DsRed 690⁻ (5′-TTGGAGTCCACGTAGTAGTAG-3′). Immediately after sacrifice, blood was removed from the rats with 200 ml intra-arterial cooled saline followed by perfusion fixation with 100 ml of 2% paraformaldehyde and 0.4% glutaraldehyde. The pancreas was cut into 0.5 cm pieces and placed into 20% sucrose solution overnight in 4° C. and then put into OTC molds at −86° C. Frozen sections 5 μm in thickness were placed on silane coated slides and fixed in 4% paraformaldehyde for 15 min at 4° C., quenched with 10 mM glycine in PBS for 5 minutes, rinsed with PBS, permeabilized with 0.5% Triton X-100 in PBS for 10 min, and rinsed with PBS for 10 min. A coverslip was anchored with a drop of nail polish at one side. The slide was then placed in aluminum ‘boat’ directly on the block of the thermocycler.) A 50 μl PCR reaction solution (0.8 units of Taq DNA polymerase, 2 μl of DsRed primers, 3 μl of DIG-dNTP, 5 μl of 10×buffer and 40 μl of water) was added to each slide and covered by the AmpliCover Disc and Clips using the Assembly Tool (Perkin Elmer) according to the manufacturer's instructions. In situ PCR was performed using Perkin-Elmer GeneAmp system 1000 as follows: after an initial hold at 94° C. (1 min), the PCR was carried out for 11 cycles (94° C. for 1 min, 54° C. for 1 min, and 72° C. for 2 min). After amplification, the slide was immersed 2×SSC for 10 min and 0.5% paraformaldehyde for 5 min and PBS for 5 min 2 times. The digoxigenin incorporated-DNA fragment was detected using a fluorescent antibody enhancer set for DIG detection (Roche) followed by histochemical staining (PCR DIG Prob Synthesis Kit (Roche Co.; Cat. NO: 1636090). First, the sections were incubated with blocking solution for 30 min to decrease the non-specific binding of the antibody to pancreas tissue. Then, the sections were incubated with 50 μl of anti-DIG solution (1:25) for 1 h at 37° C. in a moisturized chamber. Next, the slides were washed with PBS three times with shaking, each for 5 min. again the slides were incubated with 50 μl of anti-mouse-IgG-digoxigenin antibody solution (1:25) for 1 hr at 37° C. The slides were washed with PBS three times with shaking, each for 5 min again. The slides were incubated with 50 μl of anti-DIG-fluorescence solution (1:25) for 1 hr at 37° C. The slides were then washed with PBS three times with shaking, each for 5 min again. Finally, the sections were dehydrated in 70% EtOH, 95% EtOH and 100% EtOH, each for 2 min, cleared in xylene and coverslipped.

In Situ RT-PCR for Detection of DsRed mRNA. DsRed primers. A single pair of DsRed primers were used directed against the DsRed cDNA, they are DsRed 125⁺ (5′-GAGTTCATGCGCTTCAAGGTG-3′) and DsRed 690⁻ (5′-TTGGAGTCCACGTAGTAGTAG-3′). Perfusion fixed frozen sections were prepared as described above. DNase treatment was performed with 50 μl of cocktail solution (Invitrogen) (5 μl of DNase I, 5 μl of 10×DNase buffer, and 40 μl of water) on each slide, coverslipped, incubated at 25° C. overnight, and then washed with PBS 5 min 2 times.

Reverse transcription: First-strand cDNA synthesis was performed on each slide in a 50 μl total volume with 50 μl of cocktail solution (Superscript First-strand synthesis system for RT-PCR, Invitrogen kit #11904-018) (1 μl of DsRed727⁻ primers (5′-GATGGTGATGTCCTCGTTGTG-3′), 5 μl of DTT solution, 2.5 μl of dNTP, 5 μl of 10×buffer, 5 μl of 25 mM MgCl, 29 μl of water and 2.5 μl of SuperScript II RT). A coverslip was placed and the slides incubated at 42° C. for 2 hrs; washed with PBS 5 min 2 times, rinsed with 100% ETOH for 1 min and dried.

Immunohistochemistry for Detection of DsRed protein, Insulin, and Glucagon. Cryostat sections 5-8 μm in thickness were fixed in 4% paraformaldehyde for 15 min at 4° C. and quenched for 5 min with 10 mM glycine in PBS. Sections were then rinsed in PBS 3 times, and permeabilized with 0.5% Triton X-100 in PBS for 10 min. Sections were blocked with 10% goat serum at 37° C. for 1 hr and washed with PBS 3 times. The primary antibody (Sigma Co.) (1:10000 dilution in block solution) was added and incubated at 4° C. overnight. After washing with PBS three times for 5 min, the secondary antibody (Sigma Co., anti-mouse IgG conjugated with FITC) (1:500 dilution in block solution) was added and incubated for 1 hr at 37° C. Sections were rinsed with PBS for 10 min, 5 times, and then mounted.

Cell culture and transient transfection assays. INS-1 Cell lines (rat insulinoma courtesy of Newgard lab, Duke University) were maintained in cell-appropriate media. INS-1 cells were transfected with 1 μg of luciferase reporter plasmid and 0.02 μg of pTS-RL Renilla luciferase as an internal control plasmid and 3 μl Lipofectamine 2000 in 100 μl serum free DMEM each well. The cell harvest and Firefly and Renilla luciferase activities were measured 48 hours after transfection using the Dual Luciferase Assay system (Promega) and a Turner TD 20/20 luminometer.

Statistical Analysis. Differences in luciferase activity between study groups were compared by two-way ANOVA. A p value <0.05 was considered statistically significant. Post-hoc Scheffe tests were performed only when the ANOVA F values were statistically significant.

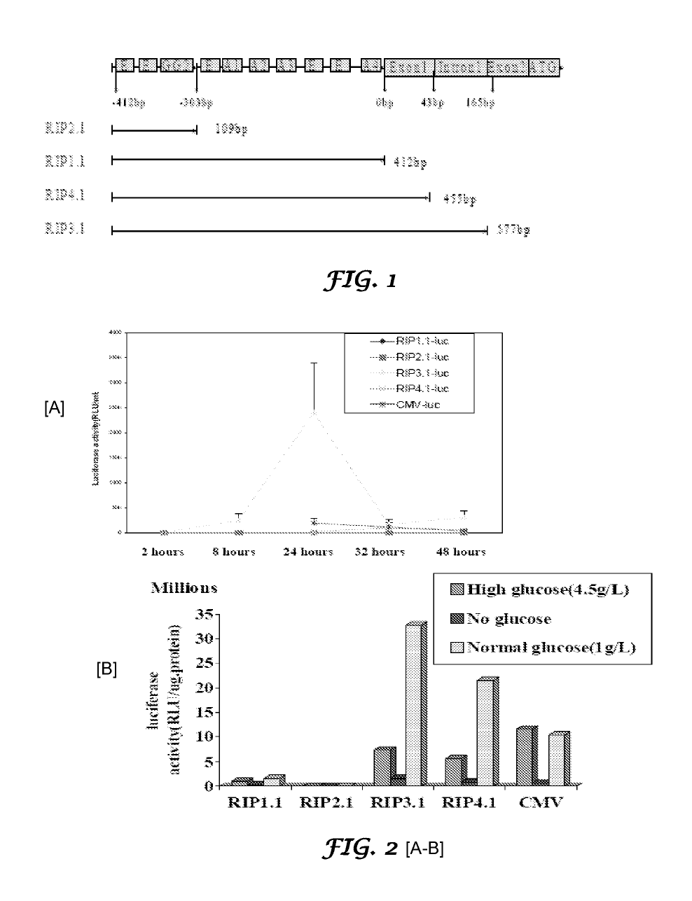

FIG. 1 is a schematic representation of the rat insulin promoter area and exon1, intron1 and exon2. The rat insulin promoter is shown with known sequence elements and fusion exon1 and exon2.

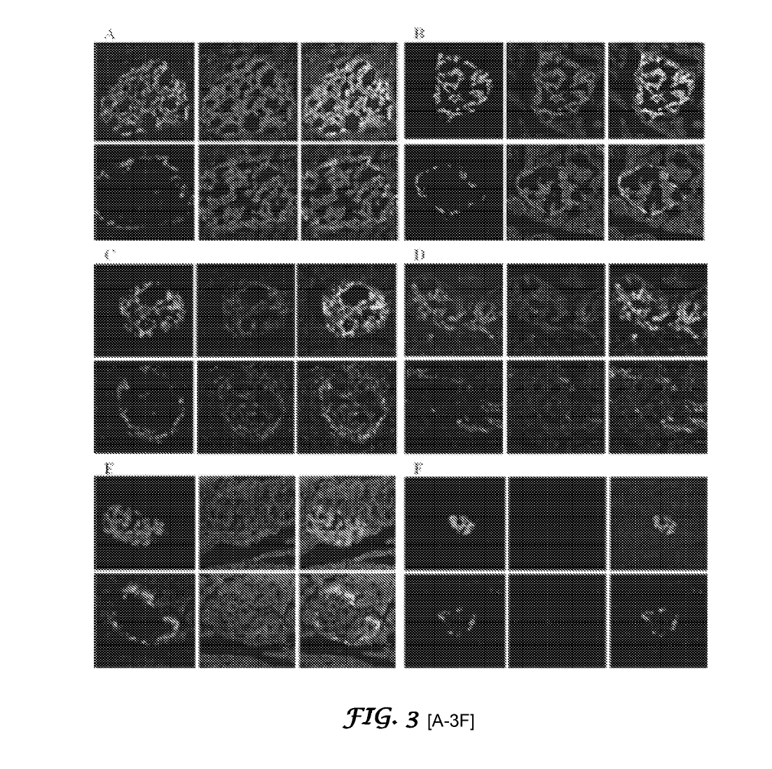

FIG. 2A-2B: top panel are luciferase activity of INS-1 cells lyses 48 hours after transfected with rips-luc under three different glucose concentration. Bottom panel are luciferase activity of culture media solution in different culture time after transfection with rips-luc under high glucose concentration, no luciferase activity in no glucose and in normal glucose concentration (data not shown).

FIG. 3A-3F. A: RIP3.1-DsRed slides, top left:green as anti-insulin; top middle red as anti-dsred; and top right as their confocal image. A bottom as sequential section and similar islets structure, bottom left: green as anti-glucagon; bottom middle:red as anti-dsred.bottom right as their confocal image. B: RIP-4.1-dsred; C: RIP-1.1-dsred; D: RIP-1.1-dsred slides, E: pCMV-dsred; F: normal control.

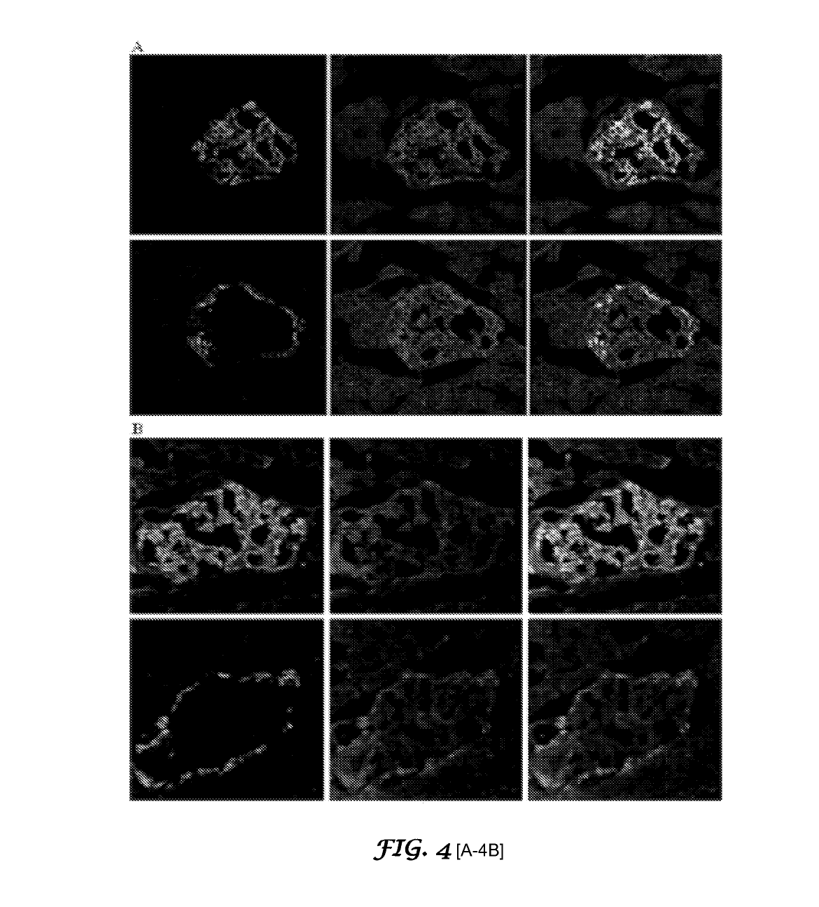

FIG. 4A-4B: A images as pRIP3.1-DsRed rats with 10% glucose feeding, A top right Green as anti-insulin; A top middle red as anti-dsred, A top left as their confocal image; A bottom right green as anti-glucagon; A bottom middle red as anti-dsred; A bottom left:their confocal image; B images as pRIP3.1-DsRed rats fasting overnight.

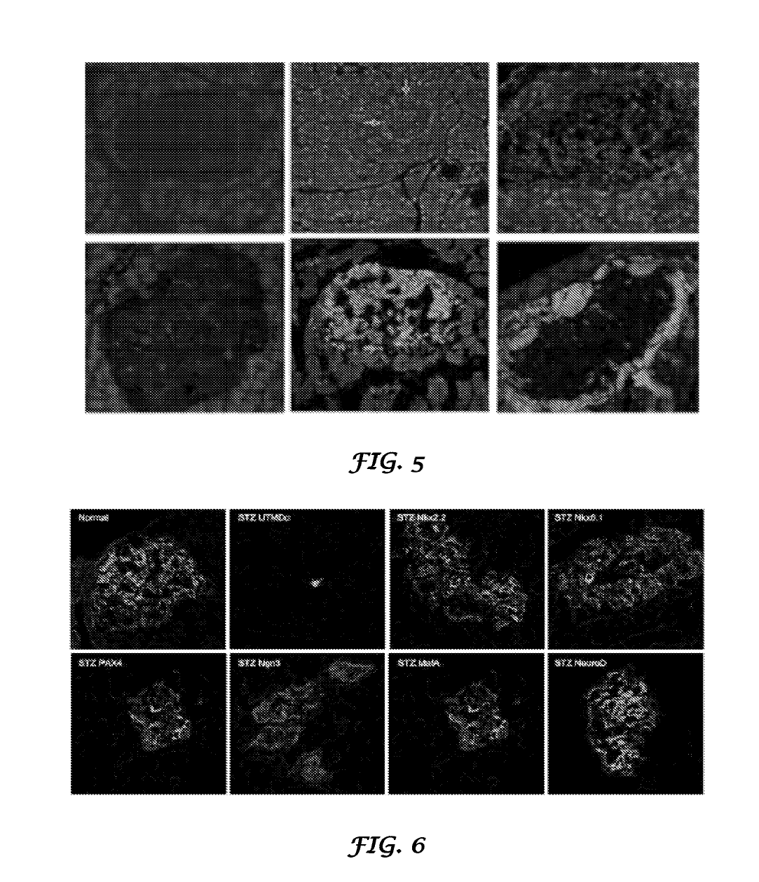

FIG. 5: Top panels. Microscopic sections (400×) from a control rat (left) and a UTMD-treated rat (middle as feeding rat and right as fasting rat). In-situ PCR was used to stain for the DsRed plasmid DNA, which is seen throughout the treated pancreas. An islet is clearly seen (arrows). Bottom panels. Sections (400×) from a control rat (left) and a rat treated with UTMD using RIP6.1-DsRed (middle as feeding rat and right as fasting rat). In-situ RT-PCR was used to stain for DsRed mRNA, which is localized to the islet center (middle/feeding). Stained at islets border (right/fasting rat).

It was found that, Rat insulin promoters driving luciferase gene transfection on rat insulinoma cell line (INS-1). Traditional rat insulin promoters driving expression in plasmids show low efficiency of tissue expression and are not highly tissue specific in in vivo delivery system. The present invention includes an insulin promoter that included insulin 1 gene exon1 and intron1 and part of exon2 not previously used for insulin gene regulation. In certain embodiments, the insulin promoter is a rat insulin promoter, a human insulin promoter or combinations thereof. FIG. 2A-2B show that under normal glucose level, luciferase activity of RIP3.1 showed a 4726-fold increase over RIP2.1 (a truncated RIP promoter), 20-fold increase over RIP1.1 (full length of traditional RIP), and even 3.1-folds of CMV-luciferase. Under no glucose conditions the gene expression of INS-1 for all constructs was significantly inhibited, luciferase activity of RIP3.1 was still 6.6 fold of RIP1.1, 2.6 fold of CMV. Under high glucose level, luciferase activities of RIP3.1 was 3515 fold of RIP2.1, 6.9 folds of RIP1.1, and 0.6 fold of CMV. Surprisingly under high glucose condition, the luciferase activity could be detected from the media solution of RIP3.1 dish at 8, 16, 24, 32 and 48 hours after transfection. No secretion was found without glucose and normal glucose culture conditions (data not shown). The RIP3.1 driving luciferase has not only highly efficient and demonstrated glucose-responsiveness it also secreted the expressed protein into the media solution in the INS-1 cell line.

RIP driving DsRed plasmids delivered to pancreatic islets of living rat with UTMD. To better understand gene expression and regulation of these RIP promoters in islets of living animals under really condition, not just in cell line, these RIP promoters driving DsRed plasmids were delivered to the pancreas of living rats with Ultrasound Targeted Microbubble Destruction (UTMD), sacrificing 4 days after UTMD. FIG. 3A-3F show that DsRed protein of RIP3.1 and RIP4.1 were detected in intact islets included islets core and border, not seen in non-islets area. Surprisingly, confocal images showed DsRed protein was detected in beta-cells and alpha-cells in islets. DsRed protein signal was much low in full length RIP1.1 and almost not seen in truncated RIP2.1. The signal of DsRed protein were seen everywhere of pancreatic slide in a CMV-DsRed plasmid treated rat. But in the normal rat control pancreas slide, no DsRed signal could be detected.

Next, it was determined whether gene expression of delivered RIP plasmids was regulatable in rat islets by glucose level, as was found with the INS-1 cell line in vitro. RIP3.1 DsRed was selected and used to treat rats, which were divided into groups: fasting (12 hours) and feeding (with 10% of glucose), and then sacrificing. FIG. 4A-4B show in the top panel (with 10% of glucose feeding) DsRed signal was seen in beta-cells and alpha-cells of islets. In the bottom panel (fasting for 12 hours) DsRed signal was only seen in alpha-cells, but not in beta-cells of islets. This indicated that glucose feeding induced upregulation of RIP3.1-DsRed gene expression in whole islets cells and fasting induced downregulation of RIP3.1-DsRed gene expression in beta-cells and kept Rip3.1-DsRed gene operating in alpha-cells.

In Situ PCR for Plasmid DNA and in Situ RT-PCR for DsRed mRNA:

FIG. 5 (top and middle 10% glucose feeding, left panel was fasting) shows the results of in situ PCR directed against RIP3.1-DsRed plasmid DNA. The plasmid DNA is seen throughout the pancreas in a nuclear pattern, including the islets. Similar patterns of homogeneous nuclear tissue localization of the plasmid were observed in the left kidney, spleen, and portions of the liver that were within the ultrasound beam. Plasmid was not present in right kidney or skeletal muscle, organs that lie outside of the ultrasound beam. Controls (top right panel)(microbubbles without plasmid or plasmid-microbubbles without ultrasound) did not show any evidence of plasmid within the pancreas. These results demonstrate that the ultrasound treatment released the plasmid within the pancreas and its immediate vicinity.

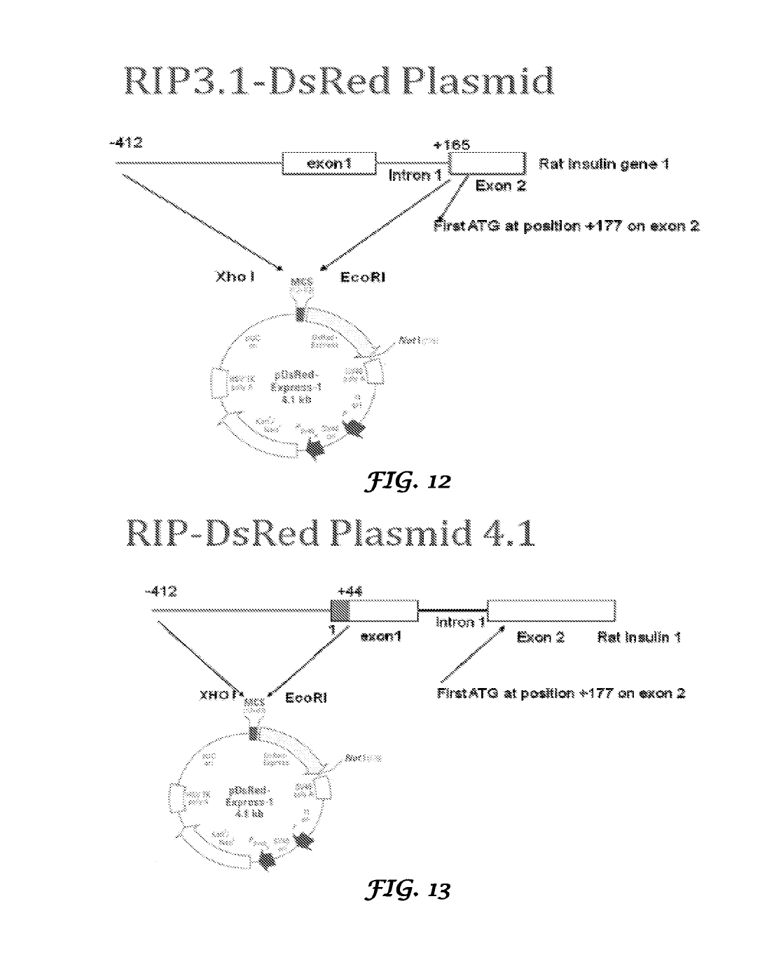

FIG. 5 (bottom middle panel (10% glucose feeding) show a representative example of in situ RT-PCR directed against the mRNA corresponding to the DsRed transcript expressed under control of the RIP3.1 promoter. DsRed mRNA was seen throughout the islets, but not in the pancreatic parenchyma, indicating that the RIP promoter directed transcription of the UTMD-delivered DsRed cDNA only in the endocrine pancreas. Bottom left panel (fasting) shows DsRed mRNA distribution in islets border area, but not in islets core. There was no signal detected in controls. Additional constructs are shown in FIGS. 11-15.

Regeneration of pancreatic islets and reversal of streptozotocin-induced diabetes by islet transcription factor genes delivered in vivo.

Blood glucose control is often inadequate in diabetes because drug therapy, including insulin replacement, is not able to replicate the glucose regulatory function of normal islets. Accordingly, new treatment strategies have focused on replenishing the deficiency of beta cell mass common to both major forms of diabetes by islet transplantation or beta-cell regeneration.^{1,2} Islet transplantation has been limited by the supply of donor islets and need for immunosuppressive therapy.³ Islet regeneration has been successful in animal models, primarily targeting liver tissue using viral vectors,⁴⁻⁷ which are not likely to be used in humans for safety reasons. The inventors have previously demonstrated that gene therapy could be targeted to pancreatic islets using ultrasound targeted microbubble destruction (UTMD).⁸ Intravenous microbubbles carrying plasmid DNA are destroyed within the pancreatic microcirculation by ultrasound, achieving local gene expression that can be further targeted to beta-cells by using the rat insulin-I promoter. UTMD has been used to deliver betacellulin and PDX1 in streptozotocin (STZ)-induced diabetes in rats with reversal of diabetes for up to 15 days by reprogramming pancreatic acinar cells into insulin-producing cells.⁹ The present invention uses UTMD to regenerate pancreatic islets using plasmids encoding NeuroD1, with normalization of blood glucose, insulin, and C-peptide for up to 30 days.

Rat insulin promoters and plasmids constructs. Sprague-Dawley rat genomic DNA was extracted from rat peripheral blood with a QIAamp Blood kit (Qiagen Inc, Valencia, Calif.) according to the manufacturer's instructions. Rat insulin gene 1 promoter fragments (−412 to +165), was amplified from SD rat genomic DNA using PCR. The resulting DNA fragments were subcloned into the pDsRed1-1 reporter vector (Clonetech, CA). hMafA cDNA and hamster Nkx6.1 cDNA were donated/cordially gifts from the Olson lab at Michigan State University and the Newgard lab at Duke University Medical Center. Rat Ngn3, NeuroD1, Pax4, and Nkx2.2 cDNAs were PCR products from Sprague-Dawley new-born rat pancreas cDNA pool that were reversed from their total RNA according to the manufacturer's instructions. Newborn rat pancreatic samples were flash frozen in liquid nitrogen and stored at −86° C. The frozen samples were thawed in 1 ml of RNA-STAT solution and immediately homogenized using a polytron homogenizer at 10,000 rpm for 30 s. Total RNA (30 ng) was reverse transcribed in 20 μl by using a Sensiscript RT kit (Qiagen Inc, Valencia, Calif.) with oligo(dT)¹⁶. The reaction mixture was incubated at 42° C. for 50 min, followed by a further incubation at 70° C. for 15 min. PCR was performed for all samples using a GeneAmp PCR System 9700 (PE ABI, Foster City, Calif., USA) in 50 ul volume containing 2 ul cDNA, 25 ul of HotStarTaq Master Mix (Qiagen Inc, Valencia, Calif.), and 20 pmol of each primer. The corresponding PCR products were verified by agarose gel electrophoresis and purified by QIAquick Gel Extraction kit (Qiagen Inc, Valencia, Calif.). To confirm the sequences, direct sequencing of PCR products was performed with dRhodamine Terminator Cycle Sequencing Kit (Applied Biosystems, Foster City, Calif.) on an ABI 3100 Genomic Analyzer. All transcriptional factor gene cDNAs were subcloned to RIP3.1 driving vector. The plasmids digestion, ligation, subclone, isolation and purification were performed by standard procedures, and once again sequenced to confirm that no artifactual mutations were present.

Animal protocols and UTMD. All animal studies were performed in accordance with National Institute of Health (NIH) recommendations and the approval of the institutional animal research committee. Male Sprague-Dawley rats (230-270 g) anesthetized with intraperitoneal ketamine (60 mg/kg) and xylazine (5 mg/kg) were shaved from left abdomen and neck, and a polyethylene tube (PE 50, Becton Dickinson, Franklin Lakes, Tenn., USA) was inserted into the right internal jugular vein by cut-down.

A total of 45 rats received one of nine treatments: (1) no treatment (normal control rats, n=3); (2) STZ (60 mg/kg/i.p., Sigma, St Louis, Mo., USA) alone without UTMD (N=3); (3) STZ and UTMD with DsRed (n=3); (4) STZ and UTMD with ngn3 (n=6); (5) STZ and UTMD with NeuroD (n=6); (6) STZ and UTMD with Pax4 (n=6). (7) STZ and UTMD with Nkx2.2 (n=6); (8) STZ and UTMD with Nkx6.1 (n=6); (9) STZ and UTMD with MafA (n=6). All genes were delivered as plasmid cDNA under the control of the RIP3.1 promoter. Blood glucose was measured 12 hours after STZ injection. Animals with fasting blood glucose over 250 mg/dl were considered as successful diabetes type 1 model and subsequently underwent UTMD within 48 hours of STZ treatment. Microbubble or control solutions (0.5 ml diluted with 0.5 ml phosphate-buffered solution (PBS)) were infused over 10 min via pump (Genie, Kent Scientific, Torrington, Conn., USA). During the infusion, ultrasound was directed to the pancreas using a commercially available ultrasound transducer (S3, Sonos 5500, Philips Ultrasound, Bothell, Wash., USA). The probe was clamped in place. Ultrasound was then applied in ultraharmonic mode (transmit 1.3 MHz/receive 3.6 MHz) at a mechanical index of 1.4. Four bursts of ultrasound were triggered to every fourth end-systole by electrocardiogram using a delay of 45-70 ms after the peak of the R wave. These settings have shown to be optimal for plasmid delivery by UTMD using this instrument.⁸ Bubble destruction was visually apparent in all rats. After UTMD, the jugular vein was tied off, the skin closed, and the animals allowed to recover. Blood samples were drawn after an overnight 12-h fast at baseline, and at different days after treatment. The protocol was repeated 3 times with rats sacrificed at days 10, 20, and 30 using an overdose of sodium pentobarbital (120 mg/kg). Pancreas, liver, spleen, and kidney were harvested for histology. Blood glucose level was measured with blood glucose test strip (Precision, Abbott, Abbott Park, Ill., USA); blood insulin, C-peptide, were measured with RIA kit (Linco Research, Radioimmunoassay, Billerica, Mass., USA).

Manufacture of Plasmid-Containing Lipid-Stabilized Microbubbles. Lipid-stabilized microbubbles were prepared as previously described in our laboratory. Briefly, a solution of DPPC (1,2-dipalmitoyl-sn-glycero-3-phosphatidylcholine, Sigma, St. Louis, Mo.) 2.5 mg/ml; DPPE (1,2-dipalmitoyl-sn-glycero-3-phosphatidylethanolamine, Sigma, St. Louis, Mo.) 0.5 mg/ml; and 10% glycerol was mixed with 2 mg of plasmid solution in a 2:1 ratio. Aliquots of 0.5 ml of this phospholipid-plasmid solution were placed in 1.5 ml clear vials; the remaining headspace was filled with the perfluoropropane gas (Air Products, Inc, Allentown, Pa.). Each vial was incubated at 4° C. for 30 min and then mechanically shaken for 30 seconds by a dental amalgamator (Vialmix™, Bristol-Myers Squibb Medical Imaging, N. Billerica, Mass.). The lipid-stabilized microbubbles appear as a milky white suspension floating on the top of a layer of liquid containing unattached plasmid DNA. The mean diameter and concentration of the microbubbles in the upper layer were measured by a particle counter (Beckman Coulter Multisizer III).

Immunohistochemistry. Cryostat sections 5-8 μm in thickness were fixed in 4% paraformaldehyde for 15 min at 4° C. and quenched for 5 min with 10 mM glycine in PBS. Sections were then rinsed in PBS 3 times, and permeabilized with 0.5% Triton X-100 in PBS for 10 min. Sections were blocked with 10% goat serum at 37° C. for 1 hr and washed with PBS 3 times. The primary antibody (mouse anti-insulin antibody, 1:500 dilution; rabbit anti-glucagon, 1:500; rabbit anti-somatostatin, 1:500; rabbit anti-pancreatic polypeptide, 1:500, rabbit anti-NeuroD1, 1:500, rabbit anti-Ki-67, rabbit anti-BrDu, 1:500 (Sigma, St. Louis, Mo.), mouse anti-ck19, 1:2000 dilution (Chemicon, Temecula, Calif.) were added and incubated for 2 hrs at RT. After washing with PBS three times for 5 min, the secondary antibody (Sigma, St; Louis, Mo.) anti-mouse IgG conjugated with FITC; anti-rabbit IgG conjugated with Cy5) (1:500 dilution in block solution) were added and incubated for 1 hr at RT. Sections were rinsed with PBS for 10 min, 5 times, and then mounted.

Data analysis. Data was analyzed with Statview software (SAS, Cary, N.C., USA). The results are expressed as mean one standard deviation. Differences were analyzed by repeated measures ANOVA with Fisher's post hoc test and considered significant at P<0.05.

The embryological development of the endocrine pancreas is associated with activation of a number of genes, encoding various transcription factors and other proteins.^{10,11} Recognizing that there may be important differences between neonatal endocrine development and islet regeneration in an adult animal with diabetes, we sought to evaluate whether the latter could be accomplished by gene therapy targeted to the pancreas by UTMD. Plasmids encoding cDNA for Ngn3, NeuroD1, Pax4, Nkx2.2, Nkx6.1, and MafA, were constructed under control of a truncated version of the rat insulin promoter (RIP3.1). Microbubbles containing these genes were infused intravenously over a 20-minute period while ultrasound was used to destroy the microbubbles within the pancreatic microcirculation. UTMD was performed 48 hours after induction of diabetes by intraperitoneal STZ (60 mg/kg). Controls included UTMD with a marker gene (RIP3.1-DsRed), STZ only without gene therapy, and normal controls that did not receive STZ. Three different repetitions of the experiments were carried out, with sacrifice at 10 days, 20 days, and 30 days to evaluate islet morphology and gene expression. Results of the 30-day studies are discussed herein and summarized in FIGS. 6-8.

FIG. 6 shows representative histological samples stained with FITC-labeled anti-insulin antibodies (green) and CY5-labeled anti-glucagon antibodies (red). These sections were obtained from a group of rats sacrificed 30 days after UTMD. Normal islets contain a central core of beta-cells (green) surrounded by a smaller number of alpha-cells (red) at the periphery of the islet. After STZ, the normal islet architecture is virtually abolished with occasional isolated beta-cells or small clusters of beta-cells remaining Gene therapy with Ngn3, Pax4, Nkx2.2, Nkx6.1, and MafA resulted in some regeneration of islets, but with abnormal islet architecture, in which alpha cells were predominant. In contrast, gene therapy with NeuroD1 resulted in regeneration of islets with nearly normal morphology. Interestingly, there were a few cells that co-stained with anti-insulin and anti-glucagon in the UTMD gene therapy groups, but not in normal islets. This finding has been previously reported to be a marker for endocrine proliferation.¹² In normal rats, there were 61±6 islets per slide, compared to only 3±2 after STZ treatment alone. NeuroD1 resulted in 37±4 islets per slide, a number that was statistically significantly higher than all other groups except normal controls (p<0.0001). The percentage of beta-cells per islet averaged 77±10% in normal controls, and was 60±6% in rats treated with NeuroD1 gene therapy. All other groups had markedly diminished numbers of beta-cells per islets. Beta-cell percentage was statistically significantly higher for NeuroD1-treated rats than for all other genes and controls (p<0.0001), but less than normal controls (p=0.0006). In addition, adjacent sections to those shown in the figures were stained with anti-somatostatin and anti-polypeptide to assess the presence of delta cells and polypeptide cells, respectively (data not shown). NeuroD1 resulted in substantial numbers of delta and polypeptide cells in the islet central core, similar to normal controls. The other transcription factors did not result in substantial numbers of delta or polypeptide cells within the islets.

FIG. 7A-7B show blood levels of glucose (top left panel), insulin (bottom left panel), and C-peptide (top right panel) at baseline, 3 days after STZ, and 30 days after STZ. Blood glucose increased dramatically by day 3 in all rats treated with STZ (approximately 400 mg/dl), and remained elevated at day 30 except in the NeuroD1-treated rats. At day 30, blood glucose was 101±11 mg/dl in the NeuroD1-treated rats, which was statistically significantly lower than all other STZ-treated groups (p<0.0001), but not from the normal controls. In the shorter-term experiments, blood glucose was also normal at days 10 and 20 in the NeuroD1-treated rats. The middle and bottom panels of FIG. 7 show that insulin and C-peptide levels were markedly depressed at day 3 in all STZ-treated rats. By day 30, insulin and C-peptide levels were nearly normal in the NeuroD1-treated rats, being statistically significantly higher than at day 3 or than all other STZ-treated groups (p<0.0001). To determine if these insulin and C-peptide levels were glucose-responsive, a separate group of 6 NeuroD1-treated rats and controls (3 normal, 3 STZ-DsRed) underwent a glucose tolerance test 30 days after UTMD. As shown in the bottom right panel of FIG. 7, the NeuroD1-treated rats had a glucose tolerance test that was nearly identical to normal controls.

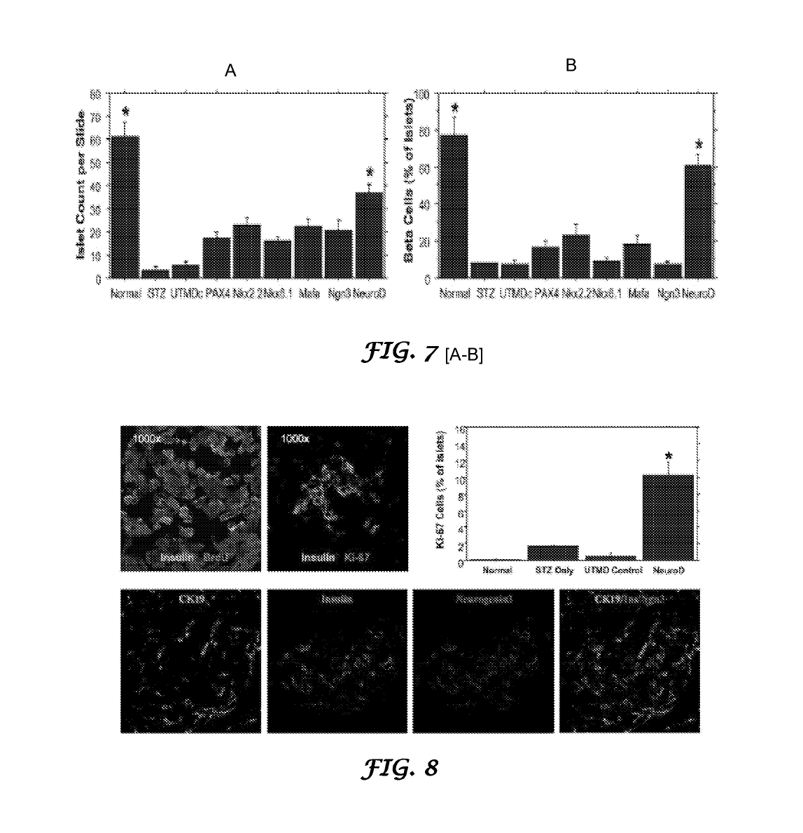

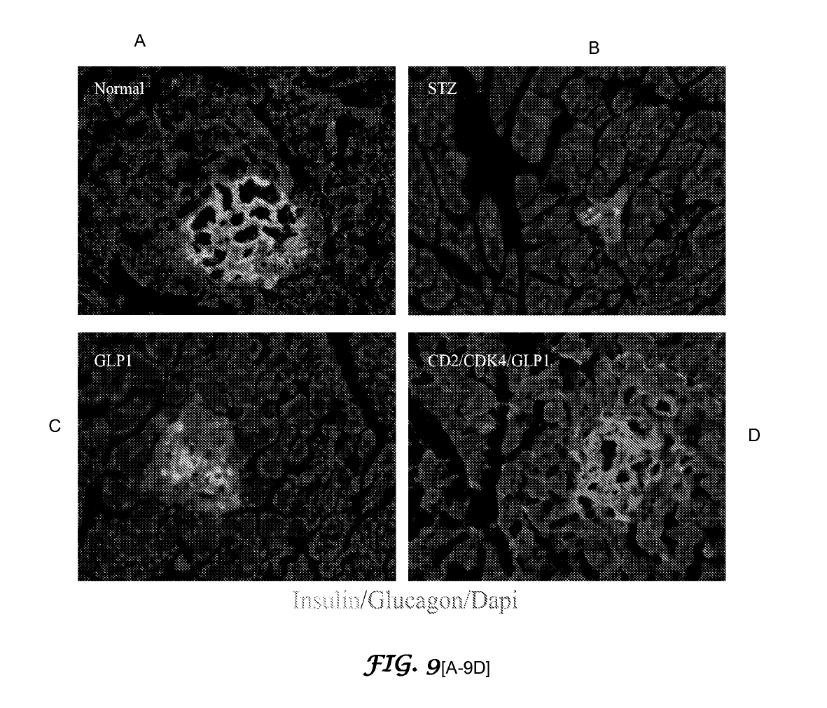

FIG. 8 shows the results of BrdU (top left) and Ki67 (top middle) staining, which indicate cellular proliferation. These are high power images of single islets from NeuroD1-treated rats. Nuclear staining with BrdU (red, top left panel) and Ki67 (red, top middle panel) are present in insulin-positive (green) cells. Compared to normal, and STZ-treated control groups, both BrdU and Ki67-positive cells were statistically significantly more numerous at 30±2% and 10±2% of insulin positive cells, respectively (p<0.0001). Normal controls had no evidence of BrdU staining, and only rare cells that were positive for Ki67. The bottom panels show immunofluorescent staining for CK19 (green, far left panel), insulin (red, left middle panel), neurogenin 3 (blue, right middle panel), and the combined image (far right panel). There is no colocalization of CK19, a ductal cell marker, with insulin or neurogenin 3; whereas as the latter two markers are colocalized within beta-cells in the islet center. This indicated that islet regeneration is not likely to be of ductal cell origin.

This study shows that in vivo delivery of NeuroD1 targeted to the pancreas of STZ-treated rats results in regeneration of nearly normal appearing islets with restoration of normal blood levels of glucose, insulin, and C-peptide. The UTMD method allows noninvasive targeting of gene delivery to the pancreas, which is the normal environment for islets. The peak expression of reporter genes delivered as plasmids by UTMD is 4 days, with rapid degradation thereafter.⁸ Thus, transient gene expression by this method is capable of inducing islet regeneration, while theoretically minimizing the risk of oncogenesis that might be associated with prolonged expression of exogenous gene therapy.

NeuroD1 is a basic helix-loop-helix transcription factor that is found in the pancreas, intestine, and central nervous system.¹³ NeuroD1 is present at pancreatic bud development and remains detectable in all mature islet cell types. In NeuroD1 knockout mice, all endocrine cell types develop, but there is decreased numbers of islets and increased beta-cell apoptosis.¹⁴ It is thought that NeuroD1 is not essential for early differentiation, but plays an important role in later stage differentiation and maintenance of beta cells, and in cell fate determination.^{15,16} This view of the role of NeuroD1 in endocrine development, though based primarily on transgenic mouse studies, is consistent with the observed finding of islet regeneration of islets containing multiple cell types in the present study.

Other transcription factors, specifically Ngn3, Pax4, Nkx2.2, Nkx6.1, and MafA, also resulted in islet regeneration, but the islets were comprised predominantly of alpha cells, and blood glucose, insulin, and C-peptide were not normalized. Interestingly, transgenic mice expressing Ngn3 under regulation of a Pdx1 promoter show mostly glucagon-positive cells,¹⁷ similar to our findings after in vivo delivery of Ngn3. When Ngn3 is overexpressed in developing chicken gut, it also produces predominantly alpha cells.¹⁸ The role of these transcription factors when delivered exogenously to adult animals with diabetes, may differ from their role in embryological development. It is also possible that various combinations of transcription factor genes or other genes implicated in pancreatic development or cell cycling, would result in even more robust islet regeneration than that observed in this study. For example, Chen, et al, showed that insulin production by acinar cells could be induced in STZ-treated rats by the combination of betacellulin and Pdx1 plasmids delivered in vivo with restoration of normal blood glucose and insulin for up to 15 days.⁹ More recently, Zhou, et al, reported that the combination of Ngn3, MafA, and Pdx1, delivered by direct injection of adenovirus into the pancreas of immune-deficient mice, resulted in reprogramming of exocrine cells to a beta-cell phenotype.¹⁹ These new beta-cells were isolated into single cells or small clusters of only a few cells, rather than aggregating into islets. Blood glucose, insulin, and C-peptide were improved but not restored to normal levels. When single transcription factors were delivered, new beta cells were not seen in significant numbers. The present study differs in at least two potentially important respects. First, a non-viral gene delivery method was used that, unlike direct injection, targets the entire pancreas. Second, a beta-cell specific promoter was used to target the endocrine pancreas. The commonly used CMV promoter is highly efficient in exocrine, but not endocrine pancreas.²⁰ Similarly, adenovirus is more robust in exocrine than endocrine pancreas.²¹

FIG. 9A-9D show images of stable islets treated with a UTMD composition comprising a combination CyclinD2, CDK4, and GLP1 (islets were stable up to 180 days) when treated with the combination. The upper left panel shows a representative islet from a normal control rat not treated by UTMD. A large dense islet core of beta cells expressing insulin is present (green) surrounded by a small capsule of peripheral alpha cells expressing glucagon (red). The upper right panel shows a representative islet remnant after STZ-induced diabetes. Only a few beat cells are present. The bottom left panel shows an example of islet regeneration after UTMD with the GLP1 gene. A smaller than normal islet is present with some beta cells (green) and alpha cells (red), but the architecture is not normal. Similar findings were present (not shown) for rats treated with UTMD using the single genes CyclinD2, CDK4, and CDK6. The bottom right panel shows a nearly normal islet after UTMD with the combination of CyclinD2, CDK4, and GLP1 (these islets were stable up to 180 days and were accompanied by reversal of diabetes with normal blood glucose, insulin and C-peptide levels).

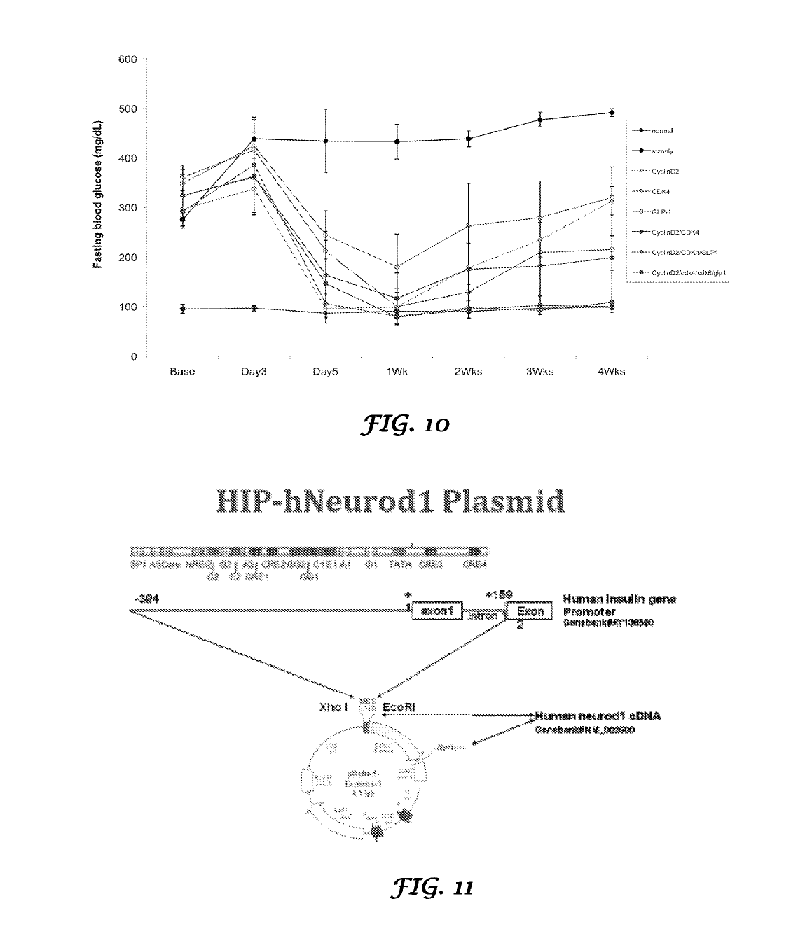

FIG. 10 is a plot showing blood glucose levels over time of islets in various groups of rats treated with UTMD gene therapy, as well as normal controls, and STZ diabetic rats without UTMD treatment. As can be seen, single gene therapy with CyclinD2, CDK4, CDK6, or GLP1 did not result in normalization of blood glucose. However, the composition comprising a combination CyclinD2, CDK4, and GLP1, or CyclinD2, CDK4, CDK6, and GLP1 restored blood glucose to normal levels for 4 weeks in this particular experiment. Longer term studies in another group of animals confirmed a duration of effect of up to 180 days.

Islet regeneration has been achieved in STZ-mediated diabetes by using adenovirus to deliver various genes to liver, with resultant restoration of normal blood glucose.⁴⁻⁶ However, adenovirus is not suitable for human use due to safety considerations. Although liver is a suitable organ for islet regeneration and islet transplantation, the present invention uses ultrasound-mediated microbubble destruction to deliver plasmid cDNA to the whole pancreas with relatively potent organ specificity.^{8,9} Targeting the pancreas offers an advantage for islet regeneration and maintenance since the pancreas is the normal physiologic milieu for islets.

The regenerated islets may represent replication of scattered beta-cells that survived the STZ-treatment. Immunohistochemical staining with Ck19, a ductal cell marker, did not show any significant uptake in the regenerated islets. Islet regeneration was only seen to occur when gene delivery was administered immediately after STZ treatment or within 48 hours afterward. When the experiments were repeated with gene delivery 7 days after STZ, a time period in which there were virtually no insulin-staining cells in STZ control rats, islet regeneration was not seen and there was progression of severe hyperglycemia and weight loss. A modification of the rat insulin I promoter was used, which is strongly beta-cell specific,⁸ and would not be expected to have substantial activity in exocrine pancreas. These considerations are consistent with the prevailing view that beta-cell replication is the predominant mechanism for increasing beta-cell mass^{12,22,23} It is interesting that stimulation of surviving beta cells with various transcription factors under control of a beta-cell specific promoter resulted in substantial numbers of alpha cells, delta cells, and polypeptide cells. This suggests that these transcription factors, especially NeuroD1, not only induce beta-cells to proliferate and aggregate into islets, but to form other islet cell types as well. The mechanism by which this occurs remains to be elucidated.

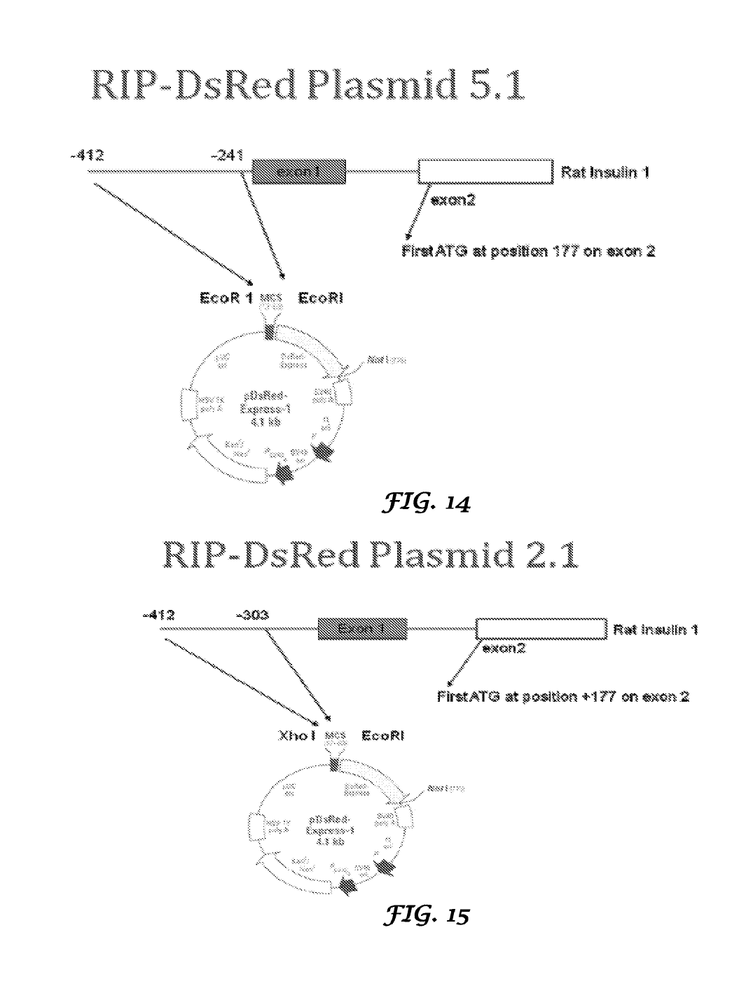

FIG. 11 is a schematic representation of a map of the HIP-hNeuroD1 plasmid showing the human insulin promoter area and exon1, intron1 and exon2 regions. FIG. 12 is a schematic representation of a map of the RIP3.1-DsRed plasmid showing the exon1, intron1 and exon2 regions. FIG. 13 is a schematic representation of a map of the RIP-DsRed 4.1 plasmid showing the rat insulin 1 and exon1, intron1 and exon2. FIG. 14 is a schematic representation of a map of the RIP-DsRed 5.1 plasmid showing the rat insulin area and exon1 and exon2. FIG. 15 is a schematic representation of a map of the RIP-DsRed 2.1 plasmid showing the rat insulin promoter area and exon1 and exon2.

The concept of using gene therapy to promote islet regeneration in diabetic patients is plausible. Meier, et al, showed that 88% of autopsy specimens from adult humans with long-standing Type I diabetes had substantial numbers of beta-cells, as well as beta-cell apoptosis and T-lymphyocyte infiltration.²⁴ Thus, existing beta-cells are a potential target for regenerative gene therapy with NeuroD1, alone or in combination with other transcription factors. However, any strategy to regenerate islets will have to also account for potential destruction of the new islets by apoptosis, inflammation, or autoimmunity. It is possible to combine regenerative genes with genes or drugs that inhibit apoptosis,²⁵⁻²⁸ and/or the autoimmune response. With regard to the latter, recent evidence suggests that tacrolimus and sirolimus might have direct toxic effects on beta cells.¹² The optimal immunosuppressive regimen is established based on the patient's needs and immune response (if any). Finally, there are important differences between rodent and human islet biology,² so these findings need to be confirmed in higher order animals before human trials can be contemplated.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

### New Vectors.

1. Newgard C B: While tinkering with the B-cell metabolic regulatory mechanisms and new therapeutic strategies: American Diabetes Association Lilly Lecture, 2001. Diabetes 51:3141-3150, 2002
2. Bonner-Weir, S, and Weir, G. C. New sources of pancreatic B-cells. Nat. Biotechnol. 23:857-861, 2005
3. Samson, S L, Chan L. Gene therapy for diabetes: reinventing the islet. Trends in endocrinology and metabolism 17:92-100, 2006
4. McClane S J, Chirmule N, Burke C V, Raper S E: Characterization of the immune response after local delivery of recombinant adenovirus in murine pancreas and successful strategies for readministration. Human Gene Ther 8:2207-2216, 1997
5. Ayuso E, Chillon M, Agudo J, Haurigot V, Bosch A, Carretero A, Otaegui P J, Bosch F: In vivo gene transfer to pancreatic beta cells by systemic delivery of adenoviral vectors. Human Gene Ther 15:805-812, 2004
6. Wang A Y, Peng P D, Ehrhardt A, Storm T A, Kay M A: Comparison of adenoviral and adeno-associated viral vectors for pancreatic gene delivery in vivo. Human Gene Ther 15:405-413, 2004
7. Gao G P, Alvira M R, Wang L, Calcedo R, Johnston J, Wilson J M: Novel adeno-associated viruses from rhesus monkeys as vectors for human gene therapy. Proc Natl Acad Sci USA 99:11854-11859, 2002
8. Gao G, Vandenberghe L H, Alvira M R, Lu Y, Calcedo R, Zhou X, Wilson J M: Clades of Adeno-associated viruses are widely disseminated in human tissues. J Virol 78:6381-6388, 2004
9. Wang Z, Ma H I, Li J, Sun L, Zhang J, Xiao X: Rapid and highly efficient transduction by double-stranded adeno-associated virus vectors in vitro and in vivo. Gene Ther 10:2105-2111, 2003
10. McCarty D M, Fu H, Monahan P E, Toulson C E, Naik P, Samulski R J: Adeno-associated virus terminal repeat (TR) mutant generates self-complementary vectors to overcome the rate-limiting step to transduction in vivo. Gene Ther 10:2112-2118, 2003

Regeneration of pancreatic islets and reversal of streptozotocin-induced diabetes by islet transcription factor genes delivered in vivo
1. Bonner-Weir, S., & Wier, G. C. New sources of pancreatic β-cells. Nat. Biotechnol. 23, 857-861 (2005).
2. Butler, P. C., Meier, J. J., Butler, A. E., & Bhushan A. The replication of β cells in normal physiology, in disease, and for therapy. Nat. Clin. Pract. Endocrinol. Metab. 3, 758-768 (2007).
3. Shapiro, A. M., et al. International trial of the Edmonton protocol for islet transplantation. New Engl. J. Med. 355, 1318-1330 (2006).
4. Ferber, S., et al. Pancreatic and duodenal homeobox gene 1 induces expression of insulin genes in liver and ameliorates streptozotocin-induced hyperglycemia. Nat. Med. 6, 568-572 (2000).
5. Kojima, H., et al. NeuroD-betacellulin gene therapy induces islet neogenesis in the liver and reverses diabetes in mice. Nat. Med. 9, 596-603 (2003).
6. Kaneto, H., et al. PDX-1/VP16 fusion protein, together with NeuroD or Ngn3, markedly induces insulin gene transcription and ameliorates glucose tolerance. Diabetes 54, 1009-1022 (2005).
7. Wang, A. Y., Ehrhardt, A., Xu, H, & Kay, M. A. Adenovirus transduction is required for the correction of diabetes using Pdx-1 or Neurogenin-3 in the liver. Mol. Ther. 15, 255-263 (2007).
8. Chen, S., et al. Efficient gene delivery to pancreatic islets with ultrasonic microbubble destruction technology. Proc. Natl. Acad. Sci. U.S.A. 103, 8469-8474 (2006).
9. Chen, S., et al. Reversal of streptozotocin-induced diabetes in rats by gene therapy with betacellulin and pancreatic duodenal homeobox-1. Gene Therapy 14, 1102-1110 (2007).
10. Scharfman, R. Control of early development of the pancreas in rodents and humans: implications of signals from the mesenchyme. Diabetologia 43, 1083-1092 (2000).
11. Bouwens, L., & Rooman, I. Regulation of pancreatic beta-cell mass. Physiol. Rev. 85, 1255-1270 (2005).
12. Nir, T., Melton, D. A., & Dor, Y. Recovery from diabetes in mice by 13 cell regeneration. J. Clin. Invest. 117, 2553-2561 (2007).
13. Naya, F. J., Stellrecht, C. M., & Tsai, M. J. Tissue-specific regulation of the insulin gene by a novel basic helix-loop-helix transcription factor. Genes Dev. 9, 1009-1019 (1995).
14. Naya F. J., et al. Diabetes, defective pancreatic morphogenesis, and abnormal enteroendocrine differentiation in BETA2/neuroD-deficient mice. Genes Dev. 11, 2323-2334 (1997).
15. Dhawan, S., Georgia, S., & Bhushan, A. Formation and regeneration of the endocrine pancreas. Curr. Opin. Cell Biol. 19, 634-645 (2007).
16. Chao, C. S., Loomis, Z. L., Lee, J. E., & Sussel, L. Genetic identification of a novel NeuroD1 function in the early differentiation of islet alpha, PP and epsilon cells. Dev. Biol. 312, 523-532 (2007).
17. Apelqvist, A., et al. Notch signalling controls pancreatic cell differentiation. Nature 400, 877-881 (1999).
18. Grapin-Botton, A., Majithia, A. R., & Melton, D. A. Key evenrs of pancreas formation are triggered in gut endoderm by ectopic expression of pancreatic regulatory genes. Genes Dev. 15, 444-454 (2001).
19. Zhou, Q., Brown, J., Kanarek, A., Rajagopal, J., & Melton, D. A. In vivo reprogramming of adult pancreatic exocrine cells to β-cells. Nature [epub ahead of print] (2008).
20. Zhan, Y., Brady, J. L., Johnston, A. M., & Lew, A. M. Predominate transgene expression in exocrine pancreas directed by the CMV promoter. D.N.A Cell Biol. 19, 639-645 (2000).
21. Wang, A. Y., Peng, P. D., Ehrhardt, A., Storm, T. A., & Kay, M. A. Comparison of adenoviral and adeno-associated viral vectors for pancreatic gene delivery in vivo. Hum. Gene Ther. 15, 405-413 (2004).
22. Dor, Y., Brown, J., Martinez, O. I., & Melton, D. A. Adult pancreatic beta-cells are formed by self-duplication rather than stem cell differentiation. Nature 429, 41-46 (2004).
23. Teta, M., Rankin, M. M., Long, S. Y., Stein, G. M., & Kushner, J. A. Growth and regeneration of adult 13 cells does not involve specialized progenitors. Dev. Cell 12, 817-826 (2007).
24. Meier, J. J., Bhushan, A., Butler, A. E., Rizza, R. A., Butler, P. C. Sustained beta cell apoptosis in patients with long-standing type I diabetes: indirect evidence for islet regeneration? Diabetologia 48, 2221-2228 (2005).
25. Dror, V., et al. Notch signaling suppresses apoptosis in adult human and mouse pancreatic islet cells. Diabetologia 50, 2504-2515 (2007).
26. Drucker, D. J. Glucagon-like peptide-1 and the islet beta-cell: augmentation of cell proliferation and inhibition of apoptosis. Endocrinology. 144, 5145-5148 (2003).
27. Noguchi, H., et al. Activation of c-Jun NH2-terminal kinase (JNK) pathway during islet transplantation and prevention of islet graft loss by intraportal injection of JNK inhibitor. Diabetologia 50, 612-619 (2007).
28. Lin, C. Y., Gurlo, T., Haataja, L., Hsuch, W. A., Butler, P. C. Activation of peroxisome proliferator-activated receptor-gamma by rosiglitazone protects human islet cells against human islet amyloid polypeptide toxicity by a phosphatidylinositol 3′-kinase-dependent pathway. J. Clin. Endocrinol. Metab. 90, 6678-6686 (2005).

What is claimed is:

1.-37. (canceled)

38. A composition for ultrasound-targeted microbubble destruction in the pancreas comprising:
a pre-assembled liposome-nucleic acid microbubble complex that comprises nucleic acid comprising GLP1, CDK4, and Cyclin D2 under the control of an insulin promoter.

39. The composition of claim 38, wherein the insulin promoter is further defined as comprising the 5′ untranslated region and exon1 of the insulin gene.

40. The composition of claim 39, wherein the insulin promoter further comprises intron 1 and exon 2 of the insulin gene.

41. The composition of claim 38, wherein the insulin promoter is further defined as comprising 50 contiguous bases upstream of the start site upstream of the NeuroD gene.

42. The composition of claim 38, wherein the complex further comprises nucleic acid encoding one or more genes selected from the group consisting of NeuroD, ngn3, PDX1, Mafa, betacellulin, Nkx2.2, Nkx6.1, PAX4, Isl1, a member of the cyclin family, a member of the cyclin dependent kinase family, and siRNA against a cyclin dependent kinase inhibitor, wherein the genes are operatively linked to the insulin promoter.

43. The composition of claim 42, wherein the cyclin dependent kinase inhibitor is a member of the INK4 family or a member of the CIP/KIP family.

44. The composition of claim 43, wherein the member of the INK4 family is p16.

45. The composition of claim 43, wherein the member of the CIP/KIP family is p27.

46. The composition of claim 38, further comprising an agent that is co-administered with the composition, wherein the agent is selected from the group consisting of an anti-apoptotic agent, an anti-inflammatory agent, a JNK inhibitor, a tacrolimus, a sirolimus, an anakinra, a Dervin polyamide, and a combination thereof.

47. The composition of claim 38, wherein the microbubble complex comprises pre-assembled liposome-nucleic acid complex liposomes.

48. The composition of claim 47, wherein the liposomes comprise 1,2-dipalmitoyl-sn-glycero-3-phosphatidylcholine and 1,2-dipalmitoyl-sn-glycero-3-phosphatidylethanolamine glycerol mixed with a plasmid.

49. The composition of claim 38, wherein the nucleic acid is in a viral or plasmid vector.

50. The composition of claim 38, wherein the pre-assembled liposome-nucleic acid complex comprises cationic lipids, anionic lipids or mixtures and combinations thereof.

51. The composition of claim 38, wherein the microbubbles are disposed in a pharmaceutically acceptable vehicle.

52. The composition of claim 38, wherein the complex further comprises nucleic acid that comprises a hexokinase gene under the control of the insulin promoter, a nucleic acid that comprises NeuroD under the control of the insulin promoter, or a combination thereof.

53. The composition of claim 38, further comprising a coating.

54. A method of treating a diabetic patient by regenerating insulin responsive cells in vivo or in situ, comprising the step of delivering an effective amount of the composition of claim 38 to the patient, wherein disruption of the microbubble in the pancreas at a target site delivers the nucleic acid into pancreas cells at the location of an ultrasound disruption, wherein cells that incorporate the nucleic acid express insulin in response to high blood glucose levels.

55. The method of claim 54, wherein the insulin promoter is further defined as comprising the 5′ untranslated region and exon1 of the insulin promoter.

56. The method of claim 55, wherein the insulin promoter further comprises intron 1 and part of exon 2 of the insulin promoter.

57. The method of claim 54, wherein the complex further comprises nucleic acid encoding one or more genes selected from the group consisting of NeuroD, ngn3, PDX1, Mafa, betacellulin, Nkx2.2, Nkx6.1, PAX4, Isl1, a member of the cyclin family, a member of the cyclin dependent kinase family, and siRNA against a cyclin dependent kinase inhibitor, wherein the genes are operatively linked to the insulin promoter.

58. An in vitro method of restoring insulin responsiveness in a target cell comprising the steps of:
obtaining a nucleic acid segment comprising GLP1, CDK4 and Cyclin D2, all of which are operatively linked to an insulin promoter that comprises a 5′ untranslated region and exon 1, transferring the nucleic acid segment into the target cell; and
maintaining the target cell under conditions effective to express GLP1, CDK4 and Cyclin D2;
wherein the expression in the target cell causes the cell to respond to high blood glucose.

59. The method of claim 58, wherein the insulin promoter further comprises intron 1 and part of exon 2 of the insulin promoter.

60. The method of claim 58, wherein the target cell is a pancreatic cell.

61. The method of claim 58, wherein the nucleic acid segment is in a viral vector or plasmid.

62. The method of claim 58, wherein the nucleic acid segment further comprises NeuroD, ngn3, PDX1, Mafa, betacellulin, Nkx2.2, Nkx6.1, PAX4, Isl1, a member of the cyclin family, a member of the cyclin dependent kinase family, and siRNA against a cyclin dependent kinase inhibitor, wherein the genes are operatively linked to the insulin promoter
